(19) United States
(12) Patent Application Publication
(10) Pub. No.: US 2021/0369724 A1
(43) Pub. Date: Dec. 2, 2021

(54) COMBINATION OF ATR KINASE INHIBITORS WITH 2,3-DIHYDROIMIDAZO[1,2-C]QUINAZOLINE COMPOUNDS

(71) Applicant: Bayer Aktiengesellschaft, Leverkusen (DE)

(72) Inventors: Antje Margret WENGNER, Berlin (DE); Gerhard SIEMEISTER, Berlin (DE); Sylvia GRÜNEWALD, Berlin (DE); Bernard HAENDLER, Berlin (DE); Ningshu LIU, Berlin (DE)

(73) Assignee: Bayer Aktiengesellschaft, Leverkusen (DE)

(21) Appl. No.: 17/285,858

(22) PCT Filed: Oct. 9, 2019
(86) PCT No.: PCT/EP2019/077319

§ 371 Date: Apr. 15, 2021

(30) Foreign Application Priority Data
Oct. 16, 2018 (EP) 18200781.5

(51) Int. Cl. A61K 31/5377; A61P 35/00

(52) U.S. Cl. CPC A61K 31/5377; A61P 35/00

(57) ABSTRACT

The present invention relates to combinations of: component A: one or more 2,3-dihydroimidazo[1,2-c]quinazoline compounds of general formula (A1) or (A2) as defined herein, or a stereoisomer, a hydrate, a solvate, or a pharmaceutically acceptable salt thereof; and component B: one or more ATR kinase inhibitor(s) as defined herein, or a stereoisomer, a tautomer, an N-oxide, a hydrate, a solvate, or a pharmaceutically acceptable salt thereof; and, optionally, component C: one or more further pharmaceutical agent(s); and, optionally, in which either or both of components A and B in any of the above-mentioned combinations are in the form of a pharmaceutical composition which is ready for use to be administered simultaneously, concurrently, separately or sequentially. The components may be administered independently of one another by the oral, intravenous, topical, local installations, intraperitoneal or nasal route.

The present invention relates to combinations of:

component A: one or more 2,3-dihydroimidazo[1,2-c]quinazoline compounds of general formula (A1) or (A2) as defined herein, or a stereoisomer, a hydrate, a solvate, or a pharmaceutically acceptable salt thereof;
and
component B: one or more ATR kinase inhibitor(s) as defined herein, or a stereoisomer, a tautomer, an N-oxide, a hydrate, a solvate, or a pharmaceutically acceptable salt thereof; and, optionally,
component C: one or more further pharmaceutical agent(s);
and, optionally,
in which either or both of components A and B in any of the above-mentioned combinations are in the form of a pharmaceutical composition which is ready for use to be administered simultaneously, concurrently, separately or sequentially. The components may be administered independently of one another by the oral, intravenous, topical, local installations, intraperitoneal or nasal route.

Another aspect of the present invention relates to the use of such combinations as described herein for the preparation of a medicament for the treatment or prophylaxis of a disease, particularly for the treatment of a hyper-proliferative disease.

Another aspect of the present invention relates to a pharmaceutical composition comprising a combination of component A and component B, and, optionally, component C, as described herein, together with one or more pharmaceutically acceptable excipient(s).

In a further aspect, the present invention relates to a kit comprising a combination of component A and component B, and, optionally, component C, as described herein.

### BACKGROUND OF THE INVENTION

Cancer is the second most prevalent cause of death in the United States, causing 450,000 deaths per year. While substantial progress has been made in identifying some of the likely environmental and hereditary causes of cancer, there is a need for additional therapeutic modalities that target cancer and related diseases. In particular there is a need for therapeutic methods for treating diseases associated with dysregulated growth/proliferation.

Cancer is a complex disease arising after a selection process for cells with acquired functional capabilities like enhanced survival/resistance towards apoptosis and a limitless proliferative potential. Thus, it is preferred to develop drugs for cancer therapy addressing distinct features of established tumors.

The PI3K signaling pathway is one of the prominent pathways that promote tumor cell survival. PI3K is activated by many cancer related receptor tyrosine kinases (e.g. PDGFR, EGFR, HER2/3, or IGF-1R), cell adhesion molecules, GPCR, and oncogenic proteins (such as Ras). The PI3K pathway activation by genetic alteration of PI3K (activation mutation and/or amplification) and/or loss-of-function of the tumor suppressor PTEN are frequently found in many tumors. Furthermore, activation of PI3K is one of the major mechanisms causing the resistance of tumors to radio-, chemo- and targeted therapeutics.

Once PI3K is activated, it catalyzes the generation of PIP3 from PIP2. The biological active PIP3 binds to the pleckstrin homology (PH) domains of PDK-1, AKT, and other PH-domain containing proteins, such as Rho and PLC. As the consequence of binding to PIP3, these proteins are translocated to the cell membrane and are subsequently activated to induce tumor cell proliferation, survival, invasion and migration.

The integrity of the genome of eukaryotic cells is secured by complex signaling pathways, referred to as the DNA damage response (DDR), and multiple DNA repair mechanisms. Upon recognizing DNA damage activation of the DDR pathways results in cell cycle arrest, suppression of general translation, induction of DNA repair, and, finally, in cell survival or cell death, depending on the cell's ability to respond to DNA damage. Proteins that directly recognize aberrant DNA structures, such as the MRE11-Rad50-Nbs1 complex recognizing DNA double strand breaks by binding to double-stranded DNA ends, or RPA (replication protein A) binding to single stranded DNA, recruit and activate the most upstream kinases of the DDR pathway, ATM (ataxia-telangiectasia mutated), ATR (ATM- and Rad3-related, UniProtKB/Swiss-Prot Q13535), and DNA-PKcs (DNA-dependent protein kinase). Whereas ATM is primarily activated by DNA double strand breaks, and DNA-PKcs is mainly involved in non-homologous end joining process of DNA repair, ATR responds to a broad spectrum of DNA damage, including double-strand breaks and lesions derived from interference with DNA replication. Major components of downstream signaling of ATM include Chk2 and p53, whereas ATR signaling involves Chk1 and cdc25. Knockout of the ATR gene in mice is embryonically lethal and ATR knockout cells develop chromosome breaks and undergo apoptosis [E. J. Brown, D. Baltimore: ATR disruption leads to chromosomal fragmentation and early embryonic lethality. Genes Dev. 14, 397-402, 2000]. In contrast, ATM is not essential for cell survival although ATM knockout cells are hypersensitive to ionizing radiation and agents which cause DNA double-strand breaks.

ATR, which forms a complex with ATRIP (ATR-interacting protein, UniProtKB/Swiss-Prot Q8WXE1) is mainly activated by long stretches of single-stranded DNA which are generated by the continuing DNA unwinding activity of helicases upon stalled replication. This replication stress with stalled replication forks may be induced by ultraviolet light, certain chemotherapeutic drugs, hydroxyurea, or aberrant oncogenic signaling resulting in increased replication initiation or origin firing. Activation of ATR results in inhibition of the cell cycle in S or G2 phase via the Chk1-cdc25 pathway and in suppression of late origin firing. The cell gains time to resolve the replication stress and, eventually, to restart replication after the source of stress has been removed. As the ATR pathway ensures cell survival after replication stress it potentially contributes to resistance to chemotherapy. Thus inhibition of ATR kinase activity could be useful for cancer treatment.

In oncogene-driven tumor cells (e.g. Ras mutation/upregulation, Myc upregulation, CyclinE overexpression) increased replication stress has been observed as compared to healthy normal cells. ATR suppression in Ras oncogene driven cells was reported to result in substantial tumor cell killing [O. Gilad, B Y Nabet, et al.: Combining ATR suppression with oncogenic Ras synergistically increases genomic instability, causing synthetic lethality or tumorigenesis in a dosage-dependent manner. Cancer Res. 70, 9693-9702, 2010].

Although ATM and ATR are principally activated by different types of DNA damage their signaling includes some cross-talk so that they can, at least partially, substitute for each other's function. This finding suggests some tumor-cell selectivity of pharmaceutical inhibition of ATR. A healthy normal cell, which has ATM and ATR pathways in parallel, arrests in G1 phase of the cell cycle upon induced DNA damage even in presence of an ATR inhibitor. In contrast, a tumor cell deficient in ATM and/or p53 signaling relies on the ATR pathway and undergoes cell death in presence of an ATR inhibitor. This suggests that ATR inhibitors may be used for the treatment of tumors with deficient ATM signaling and/or p53 function.

Details of DDR signalling and the functional role of ATM and ATR were recently reviewed in: E. Fokas, R. Prevo et al.: Targeting ATR in DNA damage response and cancer therapeutics. Cancer Treatment Rev 40, 109-117, 2014. J. M. Wagner & S. H. Kaufmann: Prospects for the use of ATR inhibitors to treat cancer. Pharmaceuticals 3, 1311-1334, 2010. D. Woods & J. J. Tuchi: Chemotherapy induced DNA damage response. Cancer Biol. Thera. 14, 379-389, 2013. A. Marechal & L. Zou: DNA damage sensing by the ATM and ATR kinases. Cold Spring Harb. Perspect. Biol. 5, a012716, 2013. M. K. Zeman & K. A. Cimprich: Causes and consequences of replication stress. Nat. Cell Biol. 16, 2-9, 2014. S. Llona-Minguez, A. Höglund et al.: Chemical strategies for development of ATR inhibitors. Exp. Rev. Mol. Med. 16, e10, 2014.

Thus ATR kinase inhibitors represent valuable compounds that should complement therapeutic options not only as single agents but also in combination with other drugs, which are used in the treatment of hyperproliferative diseases. There is an acute medical need for additional therapeutic options for the treatment of hyper-proliferative diseases.

In the prior art it is not known that either combinations of component A as defined herein and component B as defined herein, would be effective in the treatment or prophylaxis of a disease, particularly for the treatment of a hyper-proliferative disease.

### DETAILED DESCRIPTION OF THE INVENTION

Unexpectedly, and this represents a basis of the present invention, when combinations of component A as defined herein and component B as defined herein were evaluated for the treatment of a hyper-proliferative disease synergistically increased anti-tumor activity was demonstrated with these combinations for the treatment of one or more hyper-proliferative disease(s) such as prostate, liver or ovarian cancer compared to each monotherapy.

Accordingly, in accordance with one aspect, the present invention relates to a combination comprising

component A: one or more compounds of general formula (A1) or (A2) as defined herein, or a stereoisomer, a hydrate, a solvate, or a pharmaceutically acceptable salt thereof;
and
component B: one or more ATR kinase inhibitor(s) as defined herein, or a stereoisomer, a tautomer, an N-oxide, a hydrate, a solvate, or a pharmaceutically acceptable salt thereof; and, optionally,
in which either or both of said components A and B of any of the above-mentioned combinations are in the form of a pharmaceutical composition which is ready for use to be administered simultaneously, concurrently, separately or sequentially. The components may be administered independently of one another by the oral, intravenous, topical, local installations, intraperitoneal or nasal route.

In accordance with another aspect, the present invention relates to a combination comprising

component A: one or more compounds of general formula (A1) or (A2) as defined herein, or a stereoisomer, a hydrate, a solvate, or a pharmaceutically acceptable salt thereof;
and
component B: one or more ATR kinase inhibitor(s) as defined herein, or a stereoisomer, a tautomer, an N-oxide, a hydrate, a solvate, or a pharmaceutically acceptable salt thereof; and, optionally,
component C: one or more further pharmaceutical agent(s);
and, optionally,
in which either or both of components A and B in any of the above-mentioned combinations are in the form of a pharmaceutical composition which is ready for use to be administered simultaneously, concurrently, separately or sequentially. The components may be administered independently of one another by the oral, intravenous, topical, local installations, intraperitoneal or nasal route.

The combination(s) comprising component A and component B, and optionally component C, described supra are also referred to as “combination of the present invention”. Embodiments of the combination of the present invention are described infra.

Further, in accordance with another aspect, the present invention relates to a pharmaceutical composition comprising a combination of the present invention together with one or more pharmaceutically acceptable excipient(s). Embodiments of the pharmaceutical composition of the present invention are described infra.

In accordance with another aspect, the present invention relates to a kit comprising a combination of the present invention, in which optionally either or both of components A and B are in the form of a pharmaceutical composition which is ready for use to be administered simultaneously, concurrently, separately or sequentially. The components may be administered independently of one another by the oral, intravenous, topical, local installations, intraperitoneal or nasal route. Embodiments of the kit of the present invention are described infra.

In accordance with another aspect, the present invention relates to the combination of the present invention for use in the treatment or prophylaxis of a disease, preferably of a hyper-proliferative disease as defined herein.

In accordance with another aspect, the present invention relates to the use of the combination of the present invention for the preparation of a medicament for the treatment or prophylaxis of a disease, particularly of a hyper-proliferative disease as defined herein.

In accordance with another aspect, the present invention relates to a method of treatment or prophylaxis of a disease, particularly a hyper-proliferative disease as defined herein, comprising administering to a mammal in need thereof, including a human, an amount of the combination of the present invention, which is effective for the treatment or prophylaxis of said disease, particularly of said hyper-proliferative disease.

### Component A

In accordance with an embodiment of the above-mentioned aspects of the present invention, component A of the combination of the present invention comprises one or more compounds of general formula (A1):

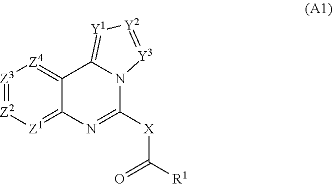

wherein
X represents CR⁵R⁶ or NH;
Y¹ represents CR³ or N;
Chemical bond between Y² [symbol] Y³ represents a single bond or double bond,
with the proviso that when the Y² [symbol] Y³ represents a double bond,
Y² and Y³ independently represent CR⁴ or N, and
when Y² [symbol] Y³ represents a single bond, Y² and Y³ independently represent CR³R⁴ or NR⁴;
Z¹, Z², Z³ and Z⁴ independently represent CH, CR² or N;
R¹ represents aryl optionally having 1 to 3 substituents selected from R¹¹, C₃₋₈cycloalkyl optionally having 1 to 3 substituents selected from R¹¹,
   C₁₋₆alkyl optionally substituted by
   aryl, heteroaryl, C₁₋₆alkoxyaryl, aryloxy, heteroaryloxy or one or more halogen,
   C₁₋₆alkoxy optionally substituted by
   carboxy, aryl, heteroaryl, C₁₋₆alkoxyaryl, aryloxy, heteroaryloxy or one or more halogen,
   or
   a 3 to 15 membered mono- or bi-cyclic heterocyclic ring that is saturated or unsaturated, and contains 1 to 3 heteroatoms selected from the group consisting of N, O and S, and optionally having 1 to 3 substituents selected from R¹¹
   wherein
   R¹¹ represents
   halogen, nitro, hydroxy, cyano, carboxy, amino, N—(C₁₋₆alkyl)amino, N-(hydroxyC₁₋₆ alkyl)amino, N,N-di(C₁₋₆alkyl)amino, N—(C₁₋₆acyl)amino, N-(formyl)-N—(C₁₋₆alkyl)amino, N—(C₁₋₆alkanesulfonyl) amino, N-(carboxyC₁₋₆alkyl)-N—(C₁₋₆alkyl)amino, N—(C₁₋₆alkoxycabonyl)amino, N—[N,N-di(C₁₋₆alkyl)amino methylene]amino, N—[N,N-di(C₁₋₆alkyl)amino (C₁₋₆alkyl)methylene]amino, N—[N,N-di(C₁₋₆alkyl)amino C₂₋₆alkenyl]amino, aminocarbonyl, N—(C₁₋₆alkyl)aminocarbonyl, N,N-di(C₁₋₆alkyl)aminocarbonyl, C₃₋₈cycloalkyl, C₁₋₆alkylthio, C₁₋₆alkanesulfonyl, sulfamoyl, C₁₋₆alkoxycarbonyl,
   N-arylamino wherein said aryl moiety is optionally having 1 to 3 substituents selected from R¹⁰¹, N-(aryl C₁₋₆alkyl)amino wherein said aryl moiety is optionally having 1 to 3 substituents selected from R¹⁰¹, aryl C₁₋₆alkoxycarbonyl wherein said aryl moiety is optionally having 1 to 3 substituents selected from R¹⁰¹,
   C₁₋₆alkyl optionally substituted by
   mono-, di- or tri-halogen, amino, N—(C₁₋₆alkyl)amino or N,N-di(C₁₋₆alkyl)amino,
   C₁₋₆alkoxy optionally substituted by
   mono-, di- or tri-halogen, N—(C₁₋₆alkyl)sulfonamide, or N-(aryl)sulfonamide,
   or
   a 5 to 7 membered saturated or unsaturated ring having 1 to 3 heteroatoms selected from the group consisting of O, S and N, and optionally having 1 to 3 substituents selected from R¹⁰¹
   wherein
   R¹⁰¹ represents
   halogen, carboxy, amino, N—(C₁₋₆alkyl)amino, N,N-di(C₁₋₆alkyl)amino, aminocarbonyl, N—(C₁₋₆alkyl)aminocarbonyl, N,N-di(C₁₋₆alkyl)aminocarbonyl, pyridyl, C₁₋₆ alkyl optionally substituted by cyano or mono- di- or tri-halogen,
   or
   C₁₋₆alkoxy optionally substituted by cyano, carboxy, amino, N—(C₁₋₆alkyl)amino, N,N-di(C₁₋₆alkyl)amino, aminocarbonyl, N—(C₁₋₆alkyl)aminocarbonyl, N,N-di(C₁₋₆alkyl)aminocarbonyl or mono-, di- or tri-halogen;
R² represents hydroxy, halogen, nitro, cyano, amino, N—(C₁₋₆alkyl)amino, N,N-di(C₁₋₆alkyl)amino, N-(hydroxyC₁₋₆alkyl)amino, N-(hydroxyC₁₋₆alkyl)-N—(C₁₋₆alkyl)amino, C₁₋₆acyloxy, aminoC₁₋₆acyloxy, C₂₋₆alkenyl, aryl,
   a 5-7 membered saturated or unsaturated heterocyclic ring having 1 to 3 heteroatoms selected from the group consisting O, S and N, and optionally substituted by
   hydroxy, C₁₋₆ alkyl, C₁₋₆ alkoxy, oxo, amino, amino C₁₋₆alkyl, N—(C₁₋₆alkyl)amino, N,N-di(C₁₋₆alkyl)amino, N—(C₁₋₆acyl)amino, N—(C₁₋₆alkyl)carbonylamino, phenyl, phenylC₁₋₆alkyl, carboxy, C₁₋₆alkoxycarbonyl, aminocarbonyl, N—(C₁₋₆alkyl)aminocarbonyl, or N,N-di(C₁₋₆alkyl)amino,
   —C(O)—R²⁰
   wherein
   R²⁰ represents C₁₋₆ alkyl, C₁₋₆ alkoxy, amino, N—(C₁₋₆alkyl)amino, N,N-di(C₁₋₆alkyl)amino, N—(C₁₋₆acyl)amino, or a 5-7 membered saturated or unsaturated heterocyclic ring having 1 to 3 heteroatoms selected from the group consisting O, S and N, and optionally substituted by
      C₁₋₆alkyl, C₁₋₆alkoxy, oxo, amino, N—(C₁₋₆alkyl)amino, N,N-di(C₁₋₆alkyl)amino, N—(C₁₋₆acyl)amino, phenyl, or benzyl,
   C₁₋₆alkyl optionally substituted by R²¹
   or
   C₁₋₆alkoxy optionally substituted by R²¹
   wherein
   R²¹ represents cyano, mono-, di or tri-halogen, hydroxy, amino, N—(C₁₋₆alkyl)amino, N,N-di(C₁₋₆alkyl)amino, N-(hydroxyC₁₋₆alkyl) amino, N-(halophenylC₁₋₆alkyl) amino, aminoC₂₋₆alkylenyl, C₁₋₆alkoxy, hydroxyC₁₋₆alkoxy, —C(O)—R²⁰¹, —NHC(O)—R²⁰¹, C₃₋₈cycloalkyl, isoindolino, phthalimidyl, 2-oxo-1,3-oxazolidinyl, aryl or a 5 or 6 membered saturated or unsaturated heterocyclic ring having 1 to 4 heteroatoms selected from the group consisting O, S and N optionally substituted by
      hydroxy, C₁₋₆alkyl, C₁₋₆alkoxy, C₁₋₆alkoxycarbonyl, hydroxyC₁₋₆ alkoxy, oxo, amino, aminoC₁₋₆alkyl, N—(C₁₋₆alkyl)amino, N,N-di(C₁₋₆alkyl)amino, N—(C₁₋₆ acyl)amino, or benzyl,
      wherein
   R²⁰¹ represents hydroxy, amino, N—(C₁₋₆alkyl)amino, N,N-di(C₁₋₆alkyl)amino, N-(halophenylC₁₋₆ alkyl) amino, C₁₋₆alkyl, aminoC₁₋₆ alkyl, aminoC₂₋₆ alkylenyl, C₁₋₆alkoxy, a 5 or 6 membered saturated or unsaturated heterocyclic ring having 1 to 4 heteroatoms selected from the group consisting O, S and N optionally substituted by
      hydroxy, C₁₋₆alkyl, C₁₋₆alkoxy, C₁₋₆alkoxycarbonyl, hydroxyC₁₋₆ alkoxy, oxo, amino, N—(C₁₋₆alkyl)amino, N,N-di(C₁₋₆alkyl)amino, N—(C₁₋₆ acyl)amino or benzyl;
R³ represents hydrogen, halogen, aminocarbonyl, or C₁₋₆alkyl optionally substituted by aryl C₁₋₆ alkoxy or mono-, di- or tri-halogen;
R⁴ represents hydrogen or C₁₋₆alkyl;
R⁵ represents hydrogen or C₁₋₆alkyl; and
R⁶ represents halogen, hydrogen or C₁₋₆alkyl;
   or a stereoisomer, a hydrate, a solvate, or a pharmaceutically acceptable salt thereof.

In accordance with an embodiment of the above-mentioned aspects of the present invention, component A of the combination of the present invention comprises one or more compounds of general formula (A1), supra, which is selected from the list consisting of specific compound Examples 1-1 to 1-210 on pp. 47 to 106, specific compound Examples 2-1 to 2-368 on pp. 107 to 204, specific compound Examples 3-1 to 3-2 on pp. 205 to 207, and specific compound Examples 4-1 to 4-2 on pp. 208 to 210, in International patent application PCT/EP2003/010377, published as WO 04/029055 A1; or a stereoisomer, a hydrate, a solvate, or a pharmaceutically acceptable salt thereof.

In accordance with an embodiment of the above-mentioned aspects of the present invention, component A of the combination of the present invention comprises 2-amino-N-[7-methoxy-8-(3-morpholin-4-ylpropoxy)-2,3-dihydroimid-azo[1,2-c]quinazolin-5-yl]pyrimidine-5-carboxamide dihydrochloride, which is hereinafter referred to as “Compound A” or “cpd. A”. It may be synthesized as described in Examples 1 and 2 of International patent application PCT/EP2012/055600, published as WO 2012/136553.

In accordance with another embodiment of the above-mentioned aspects of the present invention, component A of the combination of the present invention comprises one or more compounds of general formula (A2):

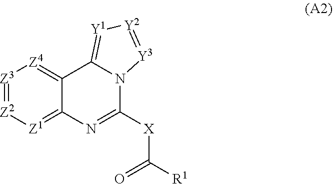

in which:
X represents CR⁵R⁶ or NH;
Y¹ represents CR³ or N;
the chemical bond between Y² [symbol] Y³ represents a single bond or double bond, with the proviso that when the Y²Y [symbol] Y³ represents a double bond, Y² and Y³ independently represent CR⁴ or N, and
when Y² [symbol] Y³ represents a single bond, Y² and Y³ independently represent CR³R⁴ or NR⁴;
Z¹, Z², Z³ and Z⁴ independently represent CH, CR² or N;
R¹ represents aryl optionally having 1 to 3 substituents selected from R¹¹, C₃₋₈cycloalkyl optionally having 1 to 3 substituents selected from R¹¹,
   C₁₋₆alkyl optionally substituted by aryl, heteroaryl, C₁₋₆alkoxyaryl, aryloxy, heteroaryloxy or one or more halogen,
   C₁₋₆alkoxy optionally substituted by carboxy, aryl, heteroaryl, C₁₋₆alkoxyaryl, aryloxy, heteroaryloxy or one or more halogen,
   or
   a 3 to 15 membered mono- or bi-cyclic heterocyclic ring that is saturated or unsaturated, optionally having 1 to 3 substituents selected from R¹¹, and contains 1 to 3 heteroatoms selected from the group consisting of N, O and S,
   wherein
   R¹¹ represents halogen, nitro, hydroxy, cyano, carboxy, amino, N—(C₁₋₆alkyl)amino, N-(hydroxyC₁₋₆alkyl)amino, N,N-di(C₁₋₆alkyl)amino, N—(C₁₋₆acyl)amino, N-(formyl)-N—(C₁₋₆alkyl)amino, N—(C₁₋₆alkanesulfonyl) amino, N-(carboxyC₁₋₆alkyl)-N—(C₁₋₆alkyl)amino, N—(C₁₋₆alkoxycabonyl)amino, N—[N,N-di(C₁₋₆alkyl)amino methylene]amino, N—[N,N-di(C₁₋₆alkyl)amino (C₁₋₆alkyl)methylene]amino, N—[N,N-di(C₁₋₆alkyl)amino C₂₋₆alkenyl]amino, aminocarbonyl, N—(C₁₋₆alkyl)aminocarbonyl, N,N-di(C₁₋₆alkyl)aminocarbonyl, C₃₋₈cycloalkyl, C₁₋₆ alkylthio, C₁₋₆alkanesulfonyl, sulfamoyl, C₁₋₆alkoxycarbonyl, N-arylamino wherein said aryl moiety is optionally having 1 to 3 substituents selected from R¹⁰¹, N-(aryl C₁₋₆alkyl)amino wherein said aryl moiety is optionally having 1 to 3 substituents selected from R¹⁰¹, aryl C₁₋₆alkoxycarbonyl wherein said aryl moiety is optionally having 1 to 3 substituents selected from R¹⁰¹,
   C₁₋₆alkyl optionally substituted by mono-, di- or tri-halogen, amino, N—(C₁₋₆alkyl)amino or N,N-di(C₁₋₆alkyl)amino,
   C₁₋₆alkoxy optionally substituted by mono-, di- or tri-halogen, N—(C₁₋₆alkyl)sulfonamide, or N-(aryl)sulfonamide,
   or
   a 5 to 7 membered saturated or unsaturated ring having 1 to 3 heteroatoms selected from the group consisting of O, S and N, and optionally having 1 to 3 substituents selected from R¹⁰¹
   wherein
   R¹⁰¹ represents halogen, carboxy, amino, N—(C₁₋₆alkyl)amino, N,N-di(C₁₋₆alkyl)amino, aminocarbonyl, N—(C₁₋₆alkyl)aminocarbonyl, N,N-di(C₁₋₆alkyl)aminocarbonyl, pyridyl,
      C₁₋₆ alkyl optionally substituted by cyano or mono- di- or tri-halogen,
      and
      C₁₋₆alkoxy optionally substituted by cyano, carboxy, amino, N—(C₁₋₆alkyl)amino, N,N-di(C₁₋₆alkyl)amino, aminocarbonyl, N—(C₁₋₆alkyl)aminocarbonyl, N,N-di(C₁₋₆alkyl)aminocarbonyl or mono-, di- or tri-halogen;
R² represents hydroxy, halogen, nitro, cyano, amino, N—(C₁₋₆alkyl)amino, N,N-di(C₁₋₆alkyl)amino, N-(hydroxyC₁₋₆alkyl)amino, N-(hydroxyC₁₋₆alkyl)-N—(C₁₋₆alkyl)amino, C₁₋₆ acyloxy, aminoC₁₋₆acyloxy, C₂₋₆alkenyl, aryl,
   a 5-7 membered saturated or unsaturated heterocyclic ring having 1 to 3 heteroatoms selected from the group consisting O, S and N, and optionally substituted by
   hydroxy, C₁₋₆alkyl, C₁₋₆alkoxy, oxo, amino, aminoC₁₋₆alkyl, N—(C₁₋₆alkyl)amino, N,N-di(C₁₋₆alkyl)amino, N—(C₁₋₆acyl)amino, N—(C₁₋₆alkyl)carbonylamino, phenyl, phenylC₁₋₆alkyl, carboxy, C₁₋₆alkoxycarbonyl, aminocarbonyl, N—(C₁₋₆alkyl)aminocarbonyl, or N,N-di(C₁₋₆alkyl)amino, —C(O)—R²⁰
   wherein
   R²⁰ represents C₁₋₆alkyl, C₁₋₆alkoxy, amino, N—(C₁₋₆alkyl)amino, N,N-di(C₁₋₆alkyl)amino, N—(C₁₋₆ acyl)amino, or a 5-7 membered saturated or unsaturated heterocyclic ring having 1 to 3 heteroatoms selected from the group consisting O, S and N, and optionally substituted by C₁₋₆alkyl, C₁₋₆alkoxy, oxo, amino, N—(C₁₋₆alkyl)amino, N,N-di(C₁₋₆alkyl)amino, N—(C₁₋₆ acyl)amino, phenyl, or benzyl,
      C₁₋₆alkyl optionally substituted by R²¹,
      or
      C₁₋₆alkoxy optionally substituted by R²¹,
      wherein
   R²¹ represents cyano, mono-, di or tri-halogen, hydroxy, amino, N—(C₁₋₆alkyl)amino, N,N-di(C₁₋₆alkyl)amino, N-(hydroxyC₁₋₆alkyl) amino, N-(halophenylC₁₋₆ alkyl)amino, aminoC₂₋₆alkylenyl, C₁₋₆alkoxy, hydroxyC₁₋₆alkoxy, —C(O)—R²⁰¹, —NHC(O)—R²⁰¹, C₃₋₈cycloalkyl, isoindolino, phthalimidyl, 2-oxo-1,3-oxazolidinyl, aryl or a 5 or 6 membered saturated or unsaturated heterocyclic ring having 1 to 4 heteroatoms selected from the group consisting O, S and N, and optionally substituted by hydroxy, C₁₋₆alkyl, C₁₋₆alkoxy, C₁₋₆alkoxycarbonyl, hydroxyC₁₋₆alkoxy, oxo, amino, aminoC₁₋₆alkyl, N—(C₁₋₆alkyl)amino, N,N-di(C₁₋₆alkyl)amino, N—(C₁₋₆ acyl)amino, or benzyl,
      wherein
   R²⁰¹ represents hydroxy, amino, N—(C₁₋₆alkyl)amino, N,N-di(C₁₋₆alkyl)amino, N-(halophenylC₁₋₆ alkyl) amino, C₁₋₆alkyl, aminoC₁₋₆alkyl, aminoC₂₋₆ alkylenyl, C₁₋₆alkoxy, a 5 or 6 membered saturated or unsaturated heterocyclic ring having 1 to 4 heteroatoms selected from the group consisting O, S and N, and optionally substituted by hydroxy, C₁₋₆alkyl, C₁₋₆alkoxy, C₁₋₆alkoxycarbonyl, hydroxyC₁₋₆alkoxy, oxo, amino, N—(C₁₋₆alkyl)amino, N,N-di(C₁₋₆alkyl)amino, N—(C₁₋₆ acyl)amino or benzyl;
R³ represents hydrogen, halogen, aminocarbonyl, or C₁₋₆alkyl optionally substituted by aryl C₁₋₆ alkoxy or mono-, di- or tri-halogen;
R⁴ represents hydrogen or C₁₋₆ alkyl;
R⁵ represents hydrogen or C₁₋₆ alkyl; and
R⁶ represents halogen, hydrogen or C₁₋₆ alkyl;
   or a stereoisomer, a hydrate, a solvate, or a pharmaceutically acceptable salt thereof.

Said compounds are published as compounds of general formulae I, Ia, Ib, Ic, Id and Ie in International patent application PCT/US2007/024985, published as WO 2008/070150 A1. These compounds of general formula I, Ia, Ib, Ic, Id and Ie are described on pp. 9 et seq., they may be synthesized according to the methods given therein on pp. 42, et seq., and are exemplified as specific compound Examples 1 to 103 therein on pp. 65 to 101. Biological test data for certain of said compounds are given therein on pp. 101 to 107.

The definitions used in relation to the compounds of formula (A1) and (A2) in this text are as follows:

The term “alkyl” refers to a straight or branched hydrocarbon chain radical consisting solely of carbon and hydrogen atoms, containing solely of carbon and hydrogen atoms, containing no unsaturation, having from one to eight carbon atoms, and which is attached to the rest of the molecule by a single bond, such as illustratively, methyl, ethyl, n-propyl 1-methylethyl (isopropyl), n-butyl, n-pentyl, and 1,1-dimethylethyl (t-butyl).

The term “alkenyl” refers to an aliphatic hydrocarbon group containing a carbon-carbon double bond and which may be a straight or branched or branched chain having about 2 to about 10 carbon atoms, e.g., ethenyl, 1-propenyl, 2-propenyl (allyl), iso-propenyl, 2-methyl-I-propenyl, 1-butenyl, 2- and butenyl.

The term “alkynyl” refers to a straight or branched chain hydrocarbonyl radicals having at least one carbon-carbon triple bond and having in the range of about 2 up to 12 carbon atoms (with radicals having in the range of about 2 up to 10 carbon atoms presently being preferred) e.g., ethynyl.

The term “alkoxy” denotes an alkyl group as defined herein attached via oxygen linkage to the rest of the molecule. Representative examples of those groups are methoxy and ethoxy.

The term “alkoxyakyl” denotes an alkoxy group as defined herein attached via oxygen linkage to an alkyl group which is then attached to the main structure at any carbon from alkyl group that results in the creation of a stable structure the rest of the molecule. Representative examples of those groups are —CH₂OCH₃ and —CH₂OC₂H₅.

The term “cycloalkyl” denotes a non-aromatic mono or multicyclic ring system of about 3 to 12 carbon atoms such as cyclopropyl, cyclobutyl, cyclopentyl, cyclohexyl and examples of multicyclic cycloalkyl groups include perhydronapththyl, adamantyl and norbornyl groups bridged cyclic group or sprirobicyclic groups e.g sprio (4,4) non-2-yl.

The term “cycloalkylalkyl” refers to cyclic ring-containing radicals containing in the range of about 3 up to 8 carbon atoms directly attached to alkyl group which is then also attached to the main structure at any carbon from the alkyl group that results in the creation of a stable structure such as cyclopropylmethyl, cyclobuyylethyl, cyclopentylethyl.

The term “aryl” refers to aromatic radicals having in the range of 6 up to 14 carbon atoms such as phenyl, naphthyl, tetrahydronapthyl, indanyl, biphenyl.

The term “arylalkyl” refers to an aryl group as defined herein directly bonded to an alkyl group as defined herein which is then attached to the main structure at any carbon from alkyl group that results in the creation of a stable structure the rest of the molecule. e.g., —CH₂C₆H₅, —C₂H₅C₆H₅.

The term “heterocyclic ring” refers to a stable 3- to 15 membered ring radical which consists of carbon atoms and from one to five heteroatoms selected from the group consisting of nitrogen, phosphorus, oxygen and sulfur. For purposes of this invention, the heterocyclic ring radical may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heteroaromatic or heteroaryl aromatic). Examples of such heterocyclic ring radicals include, but are not limited to, azetidinyl, acridinyl, benzodioxolyl, benzodioxanyl, benzofurnyl, carbazolyl cinnolinyl dioxolanyl, indolizinyl, naphthyridinyl, perhydroazepinyl, phenazinyl, phenothiazinyl, phenoxazinyl, phthalazil, pyridyl, pteridinyl, purinyl, quinazolinyl, quinoxalinyl, quinolinyl, isoquinolinyl, tetrazoyl, imidazolyl tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl pyridazinyl, oxazolyl oxazolinyl oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl quinolyl, isoquinolyl, decahydroisoquinolyl, benzimidazolyl, thiadiazolyl, benzopyranyl, benzothiazolyl, benzooxazolyl, furyl, tetrahydrofurtyl, tetrahydropyranyl, thienyl, benzothienyl, thiamorpholinyl, thiamorpholinyl sulfoxide thiamorpholinyl sulfone, dioxaphospholanyl, oxadiazolyl, chromanyl, isochromanyl.

The term “heteroaryl” refers to heterocyclic ring radical as defined herein which are aromatic. The heteroaryl ring radical may be attached to the main structure at any heteroatom or carbon atom that results in the creation of a stable structure.

The heterocyclic ring radical may be attached to the main structure at any heteroatom or carbon atom that results in the creation of a stable structure.

The term “heteroarylalkyl” refers to heteroaryl ring radical as defined herein directly bonded to alkyl group. The heteroarylalkyl radical may be attached to the main structure at any carbon atom from alkyl group that results in the creation of a stable structure.

The term “heterocyclyl” refers to a heterocylic ring radical as defined herein. The heterocylyl ring radical may be attached to the main structure at any heteroatom or carbon atom that results in the creation of a stable structure.

The term “heterocyclylalkyl” refers to a heterocylic ring radical as defined herein directly bonded to alkyl group. The heterocyclylalkyl radical may be attached to the main structure at carbon atom in the alkyl group that results in the creation of a stable structure.

The term “carbonyl” refers to an oxygen atom bound to a carbon atom of the molecule by a double bond.

The term “halogen” refers to radicals of fluorine, chlorine, bromine and iodine.

### Component B

In accordance with an embodiment of the above-mentioned aspects of the present invention, component B of the combination of the present invention comprises an ATR kinase inhibitor.

The term “ATR kinase inhibitor” as used herein means any compound that inhibits ATR kinase. Examples of such compounds are described infra.

Examples of ATR kinase inhibitors are specifically or generically disclosed in the following publications: J. Med. Chem. 2013, 56, 2125-2138; Exp. Rev. Mol. Med. 16, e10, 2014; WO2010054398A1; WO2010071837A1; WO2010073034A1; WO2011143399A1; WO2011143419A1; WO2011143422A1; WO2011143423A2; WO2011143425A2; WO2011143426A1; WO2011154737A1; WO2011163527A1; WO2012138938A1; WO2012178123A1; WO2012178124A1; WO2012178125A1; WO2013049719A1; WO2013049720A1; WO2013049722A1; WO2013049859A1; WO2013071085A1; WO2013071088A1; WO2013071090A1; WO2013071093A1; WO2013071094A1; WO2013152298A1; WO2014062604A1; WO2014089379A1; WO2014143240; WO 2014143241; WO 2014143242; ACS Med. Chem. Lett. 2015. 6, 37-41; ACS Med. Chem. Lett. 2015. 6, 42-46, WO 2015085132, WO 2015187451.

In accordance with an embodiment of the above-mentioned aspects of the present invention, component B of the combination of the present invention comprises a compound selected from VX-803, VX-970, AZD-6738 and a compound of general formula (I)

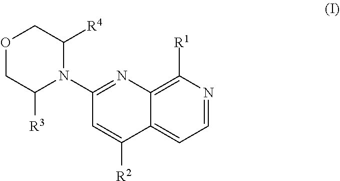

in which:
R¹ represents a group selected from:

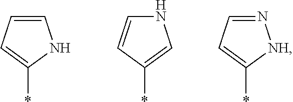 [SMILES: *c1ccc[nH]1 ; *c1cc[nH]c1 ; *c1ccn[nH]1]

wherein * indicates the point of attachment of said group with the rest of the molecule;
R² represents hydrogen, halogen, —NR⁷R⁸, CN, C₁-C₆-alkyl, C₁-C₆-alkoxy, 3- to 10-membered heterocycloalkoxy, C₂-C₆-alkenyl, C₃-C₆-cycloalkyl, 3- to 10-membered heterocycloalkyl, 4- to 10-membered heterocycloalkenyl, phenyl, heteroaryl, —(CO)OR⁷, —(CO)NR⁷R⁸, —(SO₂)R⁹, —(SO)R⁹, —SR⁹, —(SO₂)NR⁷R⁸, —NR⁷(SO₂)R⁹, —((SO)═NR¹¹)R¹⁰, —N═(SO)R⁹R¹⁰, —SiR¹⁰R¹¹R¹², —(PO)(OR⁷)₂, —(PO)(OR⁷)R¹⁰ or —(PO)(R¹⁰)₂,
   wherein each C₁-C₆-alkyl, C₁-C₆-alkoxy, 3- to 10-membered heterocycloalkoxy, C₂-C₆-alkenyl, C₃-C₆-cycloalkyl, 3- to 10-membered heterocycloalkyl, phenyl or heteroaryl is optionally substituted, one or more times, independently from each other, with halogen, OH, —NR⁷R⁸, C₁-C₆-alkyl optionally substituted one or more times with hydroxyl or phenyl, C₁-C₆-haloalkyl, C₁-C₆-alkoxy, C₃-C₆-cycloalkyl, 3- to 6-membered heterocycloalkyl, phenyl, —(CO)OR⁷, —(CO)NR⁷R⁸, —NR⁷(CO)R¹⁰, —NR⁸(CO)OR⁷, —NR⁸(CO) NR⁷R⁸, —(SO₂)R⁹, —(SO)R⁹, —SR⁹, —(SO₂)NR⁷R⁸, —NR⁷(SO₂)R⁹, —((SO)═NR¹¹)R¹⁰, —N═(SO)R⁹R¹⁰, —(PO)(OR⁷)₂, —(PO)(OR⁷)R¹⁰, —(PO)(R¹⁰)₂ or with a heteroaryl group which is optionally substituted, one or more times, with C₁-C₄-alkyl;
   wherein each 4- to 10-membered heterocycloalkenyl is optionally substituted, one or more times, independently from each other, with C₁-C₄-alkyl;
R³, R⁴ represent, independently from each other, hydrogen or methyl;
R⁷, R⁸ represent, independently from each other, hydrogen, C₁-C₆-alkyl, C₃-C₆-cycloalkyl or phenyl, which phenyl is optionally substituted, one or more times, with halogen; or
R⁷ and R⁸ together represent a 4-, 5-, 6- or 7-membered cyclic amine group, which is optionally substituted, one or more times, independently from each other, with a substituent selected from C₁-C₆-alkyl, C₁-C₆-haloalkyl, said 4-, 5-, 6- or 7-membered cyclic amine group optionally containing one further heteroatom selected from the group consisting of O, N and S;
R⁹ represents C₁-C₄-alkyl or phenyl, wherein each C₁-C₄-alkyl or phenyl is optionally substituted, one or more times, independently from each other, with R¹³;
R¹⁰ represents C₁-C₄-alkyl; or
R⁹ and R¹⁰ together, in case of —N═(SO)R⁹R¹⁰ group, represent a 5- to 8-membered heterocycloalkyl group;
R¹¹ represents hydrogen, C₁-C₄-alkyl, —(CO)OR⁷, —(CO)NR⁷R⁸ or CN;
R¹² represents hydrogen or C₁-C₄-alkyl;
R¹³ represents halogen, OH, —NR⁷R⁸, CN, NO₂, C₁-C₆-alkyl, C₁-C₆-haloalkyl, C₁-C₆-alkoxy, C₁-C₆-haloalkoxy, C₂-C₆-alkenyl, C₃-C₆-cycloalkyl, —(CO)OR⁷ or —(CO)NR⁷R⁸;
   or a stereoisomer, a tautomer, an N-oxide, a hydrate, a solvate, or a pharmaceutically acceptable salt thereof.

In context with the present invention the term “VX-803” is understood as meaning 2-amino-6-fluoro-N-[5-fluoro-4-(4-{[4-(oxetan-3-yl)piperazin-1-yl]carbonyl}piperidin-1-yl)pyridin-3-yl]pyrazolo[1,5-a]pyrimidine-3-carboxamide. It is a compound of structure:

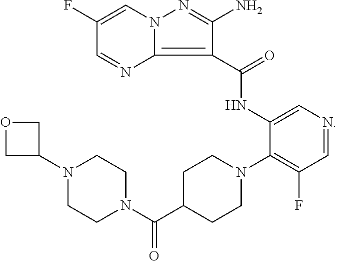 [SMILES: Nc1nn2cc(F)cnc2c1C(=O)Nc1cncc(F)c1N1CCC(C(=O)N2CCN(C3COC3)CC2)CC1]

In context with the present invention the term “VX-970” is understood as meaning 3-(3-{4-[(methylamino)methyl]phenyl}-1,2-oxazol-5-yl)-5-[4-(propan-2-ylsulfonyl)phenyl]pyrazin-2-amine. It is a compound of structure:

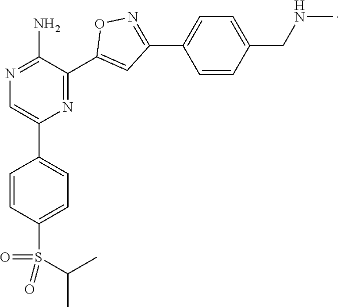 [SMILES: CNCc1ccc(-c2cc(-c3nc(-c4ccc(S(=O)(=O)C(C)C)cc4)cnc3N)on2)cc1]

In context with the present invention the term “AZD-6738” is understood as meaning 4-{4-[(3R)-3-methylmorpholin-4-yl]-6-[1-(S-methylsulfonimidoyl)cyclopropyl]pyrimidin-2-yl}-1H-pyrrolo[2,3-b]pyridine. It is a compound of structure:

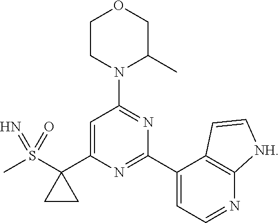 [SMILES: CC1COCCN1c1cc(C2(S(C)(=N)=O)CC2)nc(-c2ccnc3[nH]ccc23)n1]

In accordance with an embodiment of the above-mentioned aspects of the present invention, component B of the combination of the present invention comprises a compound selected from VX-803, VX-970, AZD-6738 and a compound of general formula (Ib)

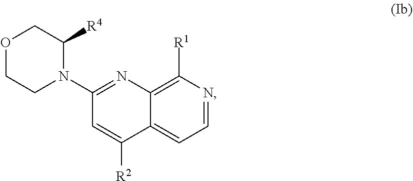

in which R¹, R², R⁴, R⁷, R⁸, R⁹, R¹⁰, R¹¹, R¹² and R¹³ are as defined for the compound of general formula (I) supra.

In accordance with an embodiment of the above-mentioned aspects of the present invention, component B of the combination of the present invention comprises a compound selected from VX-803, VX-970, AZD-6738 and a compound of general formula (Ib)

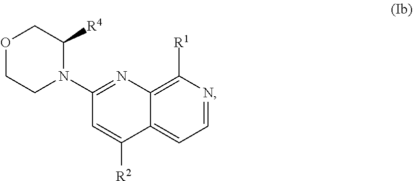

in which
R¹ represents:

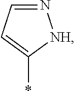 [SMILES: *c1ccn[nH]1]

wherein * indicates the point of attachment of said group with the rest of the molecule;
R² represents hydrogen, fluoro, chloro, CN, methyl, C₁-C₄-alkoxy, C₂-C₃-alkenyl, cyclopropyl, 3- to 6-membered heterocycloalkyl, 4- to 6-membered heterocycloalkenyl, phenyl, pyridinyl, thiazolyl, —(SO₂)R⁹, —SR⁹, —((SO)═NR¹¹)R¹⁰, —N═(SO)R⁹R¹⁰,
   wherein each methyl, C₁-C₄-alkoxy, C₂-C₃-alkenyl, cyclopropyl, 3- to 6-membered heterocycloalkyl, phenyl, pyridinyl or thiazolyl is optionally substituted, one or more times, independently from each other, with fluoro, chloro, OH, —NR⁷R⁸, methyl, 5-membered heterocycloalkyl, —NR⁸(CO)OR⁷, —(SO₂)R⁹, —((SO)═NR¹¹)R¹⁰—(PO)(OR⁷)₂, or with a group selected from:

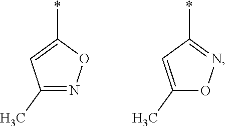

wherein * indicates the point of attachment of said group with the rest of the molecule;
wherein each 4- to 6-membered heterocycloalkenyl is optionally substituted, one or more times, with methyl;
R⁴ represents hydrogen or methyl;
R⁷, R⁸ represent, independently from each other, hydrogen or C₁-C₄-alkyl;
R⁹ represents C₁-C₄-alkyl;
R¹⁰ represents C₁-C₄-alkyl; or
R⁹ and R¹⁰ together, in case of —N═(SO)R⁹R¹⁰ group, represent a 6-membered heterocycloalkyl group;
R¹¹ represents hydrogen, methyl, —(CO)OR⁷;
   or a stereoisomer, a tautomer, an N-oxide, a hydrate, a solvate, or a pharmaceutically acceptable salt thereof.

In accordance with an embodiment of the above-mentioned aspects of the present invention, component B of the combination of the present invention comprises a compound of general formula (Ib)

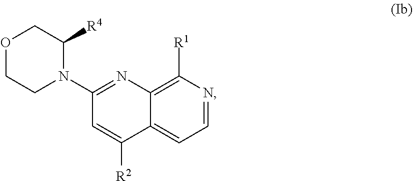

in which
R¹ represents:

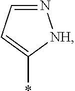 [SMILES: *c1ccn[nH]1]

wherein * indicates the point of attachment of said group with the rest of the molecule;
R² represents hydrogen, fluoro, chloro, CN, methyl, C₁-C₄-alkoxy, C₂-C₃-alkenyl, cyclopropyl, 3- to 6-membered heterocycloalkyl, 4- to 6-membered heterocycloalkenyl, phenyl, pyridinyl, thiazolyl, —(SO₂)R⁹, —SR⁹, —((SO)═NR¹¹)R¹⁰, —N═(SO)R⁹R¹⁰,
   wherein each methyl, C₁-C₄-alkoxy, C₂-C₃-alkenyl, cyclopropyl, 3- to 6-membered heterocycloalkyl, phenyl, pyridinyl or thiazolyl is optionally substituted, one or more times, independently from each other, with fluoro, chloro, OH, —NR⁷R⁸, methyl, 5-membered heterocycloalkyl, —NR⁸(CO)OR⁷, —(SO₂)R⁹, —((SO)═NR¹¹)R¹⁰, —(PO)(OR⁷)₂, or with a group selected from:

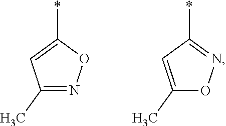

wherein * indicates the point of attachment of said group with the rest of the molecule;
wherein each 4- to 6-membered heterocycloalkenyl is optionally substituted, one or more times, with methyl;
R⁴ represents hydrogen or methyl;
R⁷, R⁸ represent, independently from each other, hydrogen or C₁-C₄-alkyl;
R⁹ represents C₁-C₄-alkyl;
R¹⁰ represents C₁-C₄-alkyl; or
R⁹ and R¹⁰ together, in case of —N═(SO)R⁹R¹⁰ group, represent a 6-membered heterocycloalkyl group;
R¹¹ represents hydrogen, methyl, —(CO)OR⁷;
   or a stereoisomer, a tautomer, an N-oxide, a hydrate, a solvate, or a pharmaceutically acceptable salt thereof.

The synthesis of the compounds of general formula (I) or (Ib) of component B is described in International Patent Publication WO2016020320 (A1).

In accordance with an embodiment of the above-mentioned aspects of the present invention, component B of the combination of the present invention comprises a compound described in one or more of the Examples of International patent application WO2016020320 (A1).

In accordance with a preferred embodiment of the above-mentioned aspects of the present invention, component B of the combination of the present invention comprises 2-[(3R)-3-methylmorpholin-4-yl]-4-(1-methyl-1H-pyrazol-5-yl)-8-(1H-pyrazol-5-yl)-1,7-naphthyridine (“Compound B”), or a stereoisomer, a tautomer, an N-oxide, a hydrate, a solvate, or a pharmaceutically acceptable salt thereof.

In a preferred embodiment, component B of the combination of the present invention is Compound B of structure

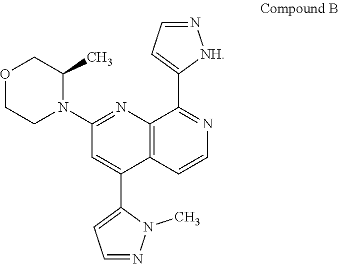 [SMILES: C[C@@H]1COCCN1c1cc(-c2ccnn2C)c2ccnc(-c3ccn[nH]3)c2n1]

The synthesis of Compound B is described in Example 111 of WO2016020320 (A1).

The definitions used in relation to the compounds of general formula (I) or (Ib) of component B in this text are as follows:

The term “halogen atom”, “halo-” or “Hal-” is to be understood as meaning a fluorine, chlorine, bromine or iodine atom.

The term “C₁-C₆-alkyl” is to be understood as meaning a linear or branched, saturated, monovalent hydrocarbon group having 1, 2, 3, 4, 5, or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl or iso-propyl group.

The term “C₁-C₆-haloalkyl” is to be understood as meaning a linear or branched, saturated, monovalent hydrocarbon group in which the term “C₁-C₆-alkyl” is defined supra, and in which one or more hydrogen atoms is replaced by a halogen atom, in identically or differently, i.e. one halogen atom being independent from another. Particularly, said halogen atom is F. Said C₁-C₆-haloalkyl group is, for example, —CF₃, —CHF₂, —CH₂F, —CF₂CF₃ or —CH₂CF₃.

The term “C₁-C₄-hydroxyalkyl” is to be understood as meaning a linear or branched, saturated, monovalent hydrocarbon group in which the term “C₁-C₄-alkyl” is defined supra, and in which one or more hydrogen atoms is replaced by a hydroxy group, e.g. a hydroxymethyl, 1-hydroxyethyl, 2-hydroxyethyl, 1,2-dihydroxyethyl, 3-hydroxypropyl, 2-hydroxypropyl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₆-alkoxy” is to be understood as meaning a linear or branched, saturated, monovalent, hydrocarbon group of formula —O-alkyl, in which the term “alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof. Particularly, said “C₁-C₆-alkoxy” can contain 1, 2, 3, 4 or 5 carbon atoms, (a “C₁-C₅-alkoxy”), preferably 1, 2, 3 or 4 carbon atoms (“C₁-C₄-alkoxy”).

The term “C₁-C₆-haloalkoxy” is to be understood as meaning a linear or branched, saturated, monovalent C₁-C₆-alkoxy group, as defined supra, in which one or more of the hydrogen atoms is replaced, in identically or differently, by a halogen atom. Particularly, said halogen atom is F. Said C₁-C₆-haloalkoxy group is, for example, —OCF₃, —OCHF₂, —OCH₂F, —OCF₂CF₃, or —OCH₂CF₃.

The term “C₂-C₆-alkenyl” is to be understood as meaning a linear or branched, monovalent hydrocarbon group, which contains one or more double bonds, and which has 2, 3, 4, 5 or 6 carbon atoms or 2, 3 or 4 carbon atoms (“C₂-C₄-alkenyl), particularly 2 or 3 carbon atoms (“C₂-C₃-alkenyl”), it being understood that in the case in which said alkenyl group contains more than one double bond, then said double bonds may be isolated from, or conjugated with, each other.

Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)ethenyl, buta-1,3-dienyl, penta-1,4-dienyl, hexa-1,5-dienyl, or methylhexadienyl group. Particularly, said group is vinyl or allyl.

The term “C₃-C₁₀-cycloalkyl” is to be understood as meaning a saturated, monovalent, mono-, or bicyclic hydrocarbon ring which contains 3, 4, 5, 6, 7, 8, 9 or 10 carbon atoms (“C₃-C₁₀-cycloalkyl”). Said C₃-C₁₀-cycloalkyl group is for example, a monocyclic hydrocarbon ring, e.g. a cyclopropyl, cyclobutyl, cyclopentyl, cyclohexyl, cycloheptyl, cyclooctyl, cyclononyl or cyclodecyl, or a bicyclic hydrocarbon ring, e.g. a perhydropentalenylene or decalin ring.

Particularly, said ring contains 3, 4, 5 or 6 carbon atoms (“C₃-C₆-cycloalkyl”), preferably cyclopropyl.

The term “3- to 10-membered heterocycloalkyl” is to be understood as meaning a saturated, monovalent, mono- or bicyclic hydrocarbon ring which contains 2, 3, 4, 5, 6, 7, 8 or 9 carbon atoms, and one or more heteroatom-containing groups selected from C(═O), O, S, S(═O), S(═O)₂, NRₐ, in which Rₐ represents a hydrogen atom, or a C₁-C₆-alkyl or C₁-C₆-haloalkyl group; it being possible for said heterocycloalkyl group to be attached to the rest of the molecule via any one of the carbon atoms or, if present, the nitrogen atom.

Particularly, said 3- to 10-membered heterocycloalkyl can contain 2, 3, 4, or 5 carbon atoms, and one or more of the above-mentioned heteroatom-containing groups (a “3- to 6-membered heterocycloalkyl”), more particularly said heterocycloalkyl can contain 4 or 5 carbon atoms, and one or more of the above-mentioned heteroatom-containing groups (a “5- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, pyrrolinyl, or a 6-membered ring, such as tetrahydropyranyl, piperidinyl, morpholinyl, dithianyl, thiomorpholinyl, piperazinyl, or trithianyl, or a 7-membered ring, such as a diazepanyl ring, for example. Optionally, said heterocycloalkyl can be benzo fused. Preferably, the 3- to 6-membered heterocycloalkyl is a tetrahydrofuranyl, tetrahydropyranyl or piperazinyl.

Said heterocycloalkyl can be bicyclic, such as, without being limited thereto, a 5,5-membered ring, e.g. a hexahydrocyclopenta[c]pyrrol-2(1H)-yl ring, or a 5,6-membered bicyclic ring, e.g. a hexahydropyrrolo[1,2-a]pyrazin-2(1H)-yl ring.

As mentioned supra, said nitrogen atom-containing ring can be partially unsaturated, i.e. it can contain one or more double bonds, such as, without being limited thereto, a 2,5-dihydro-1H-pyrrolyl, 4H-[1,3,4]thiadiazinyl, 4,5-dihydrooxazolyl, or 4H-[1,4]thiazinyl ring, for example, or, it may be benzo-fused, such as, without being limited thereto, a dihydroisoquinolinyl ring, for example.

The term “3- to 10-membered heterocycloalkoxy” of formula —O-heterocycloalkyl, in which the term “heterocycloalkyl” is defined supra, is to be understood as meaning a saturated, monovalent, mono- or bicyclic hydrocarbon ring which contains 2, 3, 4, 5, 6, 7, 8 or 9 carbon atoms, and one or more heteroatom-containing groups selected from C(═O), O, S, S(═O), S(═O)₂, NRₐ, in which Rₐ represents a hydrogen atom, a C₁-C₆-alkyl or C₁-C₆-haloalkyl group and which is connected to the rest of the molecule via an oxygen atom, e.g. a pyrrolidineoxy, tetrahydrofuraneoxy or tetrahydropyranoxy.

The term “4- to 10-membered heterocycloalkenyl” is to be understood as meaning an unsaturated, monovalent, mono- or bicyclic hydrocarbon ring which contains 3, 4, 5, 6, 7, 8 or 9 carbon atoms, and one or more heteroatom-containing groups selected from C(═O), O, S, S(═O), S(═O)₂, NRₐ, in which Rₐ represents a hydrogen atom, or a C₁-C₆-alkyl or C₁-C₆-haloalkyl group; it being possible for said heterocycloalkenyl group to be attached to the rest of the molecule via any one of the carbon atoms or, if present, the nitrogen atom. Examples of said heterocycloalkenyl may contain one or more double bonds, e.g. 4H-pyranyl, 2H-pyranyl, 3,6-dihydro-2H-pyran-4-yl, 3,6-dihydro-2H-thiopyran-4-yl, 1,2,3,6-tetrahydropyridin-4-yl, 3H-diazirinyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, 4H-[1,4]thiazinyl or 5,6-dihydroimidazo[1,2-a]pyrazin-7(8H)-yl group or it may be benzo fused.

The term “heteroaryl” is understood as meaning a monovalent, monocyclic-, bicyclic- or tricyclic aromatic ring system having 5, 6, 7, 8, 9, 10, 11, 12, 13 or 14 ring atoms (a “5- to 14-membered heteroaryl” group), 5 or 6 or 9 or 10 ring atoms (a “5- to 10-membered heteroaryl” group) or particularly 5 or 6 ring atoms (“5- to 6-membered heteroaryl” group), and which contains at least one heteroatom which may be identical or different, said heteroatom being such as oxygen, nitrogen or sulfur, and in addition in each case can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, oxepinyl or 1H-pyrrolo[2,3-b]pyridin-4-yl, etc.

In general, and unless otherwise mentioned, the heteroarylic or heteroarylenic radicals include all the possible isomeric forms thereof, e.g. the positional isomers thereof. Thus, for some illustrative non-restricting example, the term pyridinyl or pyridinylene includes pyridin-2-yl, pyridin-2-ylene, pyridin-3-yl, pyridin-3-ylene, pyridin-4-yl and pyridin-4-ylene; or the term thienyl or thienylene includes thien-2-yl, thien-2-ylene, thien-3-yl and thien-3-ylene.

### Other Definitions

Other definitions of terms used in this text, particularly in relation to components A (compounds of formula (A1) or (A2)) and component B (compounds of formula (I) or (Ib), are as follows:

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

Should a composite substituent be composed of more than one parts, e.g. (C₁-C₃-alkoxy)-(C₁-C₆-alkyl)-, it is possible for the position of a given part to be at any suitable position of said composite substituent, i.e. the C₁-C₃-alkoxy part can be attached to any carbon atom of the C₁-C₆-alkyl part of said (C₁-C₃-alkoxy)-(C₁-C₆alkyl)-group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The term “C₁-C₆”, as used throughout this text, e.g. in the context of the definition of “C₁-C₆-alkyl”, “C₁-C₆-haloalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅; particularly C₁-C₂, C₁-C₃, C₁-C₄, C₁-C₅, C₁-C₆; more particularly C₁-C₄; in the case of “C₁-C₆-haloalkyl” or “C₁-C₆-haloalkoxy” even more particularly C₁-C₂.

Similarly, as used herein, the term “C₂-C₆”, as used throughout this text, e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₆”, as used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₆, C₅-C₆; particularly C₃-C₆.

Further, as used herein, the term “C₂-C₄”, as used throughout this text, e.g. in the context of “C₂-C₄-alkenyl” is to be understood as meaning a alkenyl group having a finite number of carbon atoms of 2 to 4, i.e. 2, 3 or 4 carbon atoms. It is to be understood further that said term “C₂-C₄” is to be interpreted as any sub-range comprised therein, e.g. C₂-C₄, C₂-C₃, C₃-C₄.

For example:

“C₁-C₈” encompasses C₁, C₂, C₃, C₄, C₅, C₆, C₇, C₈, C₁-C₈, C₁-C₇, C₁-C₆, C₁-C₅, C₁- C₄, C₁-C₃, C₁-C₂, C₂-C₈, C₂-C₇, C₂-C₆, C₂-C₅, C₂-C₄, C₂-C₃, C₃-C₈, C₃-C₇, C₃-C₆, C₃-C₅, C₃-C₄, C₄-C₈, C₄-C₇, C₄-C₆, C₄-C₅, C₅-C₈, C₅-C₇, C₅-C₆, C₆-C₈, C₆-C₇ and C₇-C₈;
“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;
“C₁-C₄” encompasses C₁, C₂, C₃, C₄, C₁-C₄, C₁-C₃, C₁-C₂, C₂-C₄, C₂-C₃ and C₃-C₄;
“C₁-C₃” encompasses C₁, C₂, C₃, C₁-C₃, C₁-C₂ and C₂-C₃;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

In the context of the present invention, the term “treatment” or “treating” includes inhibition, retardation, checking, alleviating, attenuating, restricting, reducing, suppressing, repelling or healing of a disease or the development, the course or the progression of such states and/or the symptoms of such states. The term “disease” includes but is not limited a condition, a disorder, an injury or a health problem. The term “therapy” is understood here to be synonymous with the term “treatment”. The term “prophylaxis” refers to the avoidance or reduction of the risk of contracting, experiencing, suffering from or having a disease or a development or advancement of such states and/or the symptoms of such states. The treatment or prophylaxis of a disease may be partial or complete.

### Combination

In accordance with an embodiment, the present invention relates to a combination of component A and component B, wherein

said component A is 2-amino-N-[7-methoxy-8-(3-morpholin-4-ylpropoxy)-2,3-dihydroimidazo[1,2-c]quinazolin-5-yl]pyrimidine-5-carboxamide, or 2-amino-N-[7-methoxy-8-(3-morpholin-4-ylpropoxy)-2,3-dihydroimid-azo[1,2-c]quinazolin-5-yl]pyrimidine-5-carboxamide dihydrochloride;
or a solvate, hydrate or stereoisomer thereof;
optionally in the form of a pharmaceutical composition which is ready for use to be administered simultaneously, concurrently, separately or sequentially.

In accordance with an embodiment, the present invention relates to a combination of component A and component B, wherein

said component B is 2-[(3R)-3-methylmorpholin-4-yl]-4-(1-methyl-1H-pyrazol-5-yl)-8-(1H-pyrazol-5-yl)-1,7-naphthyridine;
or a stereoisomer, a tautomer, an N-oxide, a hydrate, a solvate, or a pharmaceutically acceptable salt thereof;
optionally in the form of a pharmaceutical composition which is ready for use to be administered simultaneously, concurrently, separately or sequentially.

In accordance with an embodiment, the present invention relates to combinations of component A and component B, wherein:

said component A is 2-amino-N-[7-methoxy-8-(3-morpholin-4-ylpropoxy)-2,3-dihydroimidazo[1,2-c]quinazolin-5-yl]pyrimidine-5-carboxamide, or 2-amino-N-[7-methoxy-8-(3-morpholin-4-ylpropoxy)-2,3-dihydroimid-azo[1,2-c]quinazolin-5-yl]pyrimidine-5-carboxamide dihydrochloride;
or a solvate, hydrate or stereoisomer thereof;
optionally in the form of a pharmaceutical composition which is ready for use to be administered simultaneously, concurrently, separately or sequentially;
and wherein:
said component B is 2-[(3R)-3-methylmorpholin-4-yl]-4-(1-methyl-1H-pyrazol-5-yl)-8-(1H-pyrazol-5-yl)-1,7-naphthyridine;
or a stereoisomer, a tautomer, an N-oxide, a hydrate, a solvate, or a pharmaceutically acceptable salt thereof;
optionally in the form of a pharmaceutical composition which is ready for use to be administered simultaneously, concurrently, separately or sequentially.

In accordance with an embodiment, the present invention relates to combinations of component A and component B, wherein:

said component A is 2-amino-N-[7-methoxy-8-(3-morpholin-4-ylpropoxy)-2,3-dihydroimidazo[1,2-c]quinazolin-5-yl]pyrimidine-5-carboxamide, and wherein:
said component B is 2-[(3R)-3-methylmorpholin-4-yl]-4-(1-methyl-1H-pyrazol-5-yl)-8-(1H-pyrazol-5-yl)-1,7-naphthyridine.

In accordance with an embodiment, the present invention relates to combinations of component A and component B, wherein:

said component A is 2-amino-N-[7-methoxy-8-(3-morpholin-4-ylpropoxy)-2,3-dihydroimidazo[1,2-c]quinazolin-5-yl]pyrimidine-5-carboxamide dihydrochloride, and wherein:
said component B is 2-[(3R)-3-methylmorpholin-4-yl]-4-(1-methyl-1H-pyrazol-5-yl)-8-(1H-pyrazol-5-yl)-1,7-naphthyridine.

In accordance with an embodiment, the present invention relates to a combination of any component A mentioned herein with any component B mentioned herein.

In a particular embodiment, the present invention relates to a combination of a component A with a component B, as mentioned in the Experimental Section herein.

### Useful Forms of Components A and B

As mentioned supra, either or both of the compounds of components A and B of any of the combinations of the present invention may be in a useful form, such as pharmaceutically acceptable salts, co-precipitates, metabolites, hydrates, solvates and prodrugs.

The term “pharmaceutically acceptable salt” refers to a relatively non-toxic, inorganic or organic acid addition salt of a compound of component A or B of the combination of the invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and chorine salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts of acidic compounds of component A or B of the combination of the invention are prepared by reacting said acidic compounds with the appropriate base via a variety of known methods.

The present invention includes all possible salts of the components of the combination of the present invention as single salts, or as any mixture of said salts, in any ratio.

Representative salts of the compounds of component A or B, optionally C, of the combination of the invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, chloride, bromide, iodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, sulfate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, or butyl chlorides, bromides and iodides; dialkyl sulfates like dimethyl, diethyl, dibutyl sulfate, or diamyl sulfates, long chain halides such as decyl, lauryl, myristyl and strearyl chlorides, bromides and iodides, aralkyl halides like benzyl and phenethyl bromides and others.

The compounds of the combination of the present invention can exist as a hydrate, or as a solvate, wherein the compounds of the present combination contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present combination includes all such hydrates or solvates.

Furthermore, the present invention includes all possible crystalline forms, or polymorphs, of the compounds of the components of the combination of the present invention, either as single polymorphs, or as a mixture of more than one polymorph, in any ratio.

The combination of the present invention also includes all suitable isotopic variations of the compound of component A, particularly of Compound A, and/or of the compound of component B, particularly of Compound B. An isotopic variation of the compound of component A or of the compound of component B is defined as one in which at least one atom is replaced by an atom having the same atomic number but an atomic mass different from the atomic mass usually or predominantly found in nature. Examples of isotopes that can be incorporated into the compound of components A or B include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of the compound of components A or B, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of the compound of components A or B can generally be prepared by conventional procedures known by a person skilled in the art such as by the illustrative methods or by the preparations described in the examples hereafter using appropriate isotopic variations of suitable reagents.

Preferred compounds of component A or component B are those which produce the more desirable biological activity, most preferred are Compound A and Compound B.

Separated, pure or partially purified isomers and stereoisomers or racemic or diastereomeric mixtures of the compounds of components A or B are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of component A or B of the combination of the invention can likewise be obtained by chiral syntheses utilizing optically active starting materials.

In order to limit different types of isomers from each other reference is made to IUPAC Rules Section E (Pure Appl Chem 45, 11-30, 1976).

The combination of the present invention includes all possible stereoisomers of the compounds of components A or B as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a compound of component A or component B may be achieved by any suitable state of the art method, such as chromatography, especially chiral chromatography, for example.

Further, the compounds of component A, particularly of Compound A, or component B, particularly of Compound B, may exist as tautomers. For example, any compound of component A which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, namely:

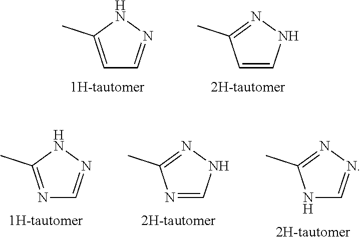

The present combination includes all possible tautomers of the compounds of component A or of component B, particularly the 1H-tautomer or the 2H-tautomer of the pyrazol-5-yl group in 8-position of the naphthyridine core of Compound B, as single tautomers, or as any mixture of said tautomers, in any ratio.

Further, the compounds of component A or component B, particularly Compound B, can exist as N-oxides, which are defined in that at least one nitrogen of the compounds of the present invention is oxidised. The present combination includes all such possible N-oxides of component A or component B.

Where the plural form of the word compounds, salts, polymorphs, hydrates, solvates, stereoisomers, tautomers, N-oxides, and the like, is used herein, this is taken to mean also a single compound, salt, polymorph, isomer, hydrate, solvate, stereoisomer, tautomer, N-oxide or the like.

### Optional Component C

The combinations of component A and component B of this invention can be administered as the sole pharmaceutical agent or in combination with one or more further pharmaceutical agents where the resulting combination of components A, B and C causes no unacceptable adverse effects. For example, the combinations of components A and B of this invention can be combined with component C, i.e. one or more further pharmaceutical agent(s), such as known anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agents, and the like, as well as with admixtures and combinations thereof

Optional pharmaceutical agent(s) which can be added as component C to the combination of components A and B can be one or more pharmaceutical agent(s) such as 131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, crisantaspase, crizotinib, cyclophosphamide, cyproterone, cytarabine, dacarbazine, dactinomycin, daratumumab, darbepoetin alfa, dabrafenib, darolutamide, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Generally, the use of pharmaceutical agent(s) as component C in combination with a combination of components A and B of the present invention will serve to:
(1) yield better efficacy in reducing the growth of a tumor and/or metastasis or even eliminate the tumor and/or metastasis as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

### Pharmaceutical Composition

The present invention relates to a pharmaceutical composition comprising a combination of the present invention together with one or more pharmaceutically acceptable excipient(s).

Further, the present invention relates to a pharmaceutical composition comprising the combination of the invention, particularly comprising Compound A and Compound B, together with one or more pharmaceutically acceptable excipient(s).

The present invention relates to a pharmaceutical composition comprising the combination of the invention, particularly comprising Compound A and Compound B and component C, together with one or more pharmaceutically acceptable excipient(s).

In another embodiment the components A and B, and optionally component C, are present in separate pharmaceutical compositions.

In another embodiment the components A and B, and optionally component C, are present in a joint formulation.

Components A and B, optionally C, of the combination of the present invention may, independently from one another, be in the form of a pharmaceutical composition which is ready for use to be administered simultaneously, concurrently, separately or sequentially. The components may be administered independently of one another by the oral, intravenous, topical, local installations, intraperitoneal or nasal route.

Said pharmaceutical compositions can be utilized to achieve the desired pharmacological effect by administration to a patient in need thereof. A patient, for the purpose of this invention, is a mammal, including a human, in need of treatment for the particular condition or disease. Therefore, the present invention includes combinations in which components A and B, optionally C, independently of one another, are pharmaceutical compositions that are comprised of a pharmaceutically acceptable carrier and a pharmaceutically effective amount of a said component. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of component, and/or combination. A pharmaceutically effective amount of a combination is preferably that amount which produces a result or exerts an influence on the particular condition being treated. The combinations of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

It is possible for the compounds of the combinations according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds of the components of the combination of the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixture agitandae), lipophilic suspensions, emulsions, ointments, creams, transdermal therapeutic systems (such as, for example, patches), milk, pastes, foams, dusting powders, implants or stents.

The compounds of the combinations according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

### Kit

In another embodiment the present invention relates to a kit comprising the combination of the invention.

In the kit optionally either or both of said components A and B in any of the above-mentioned combinations of the invention are in the form of a pharmaceutical composition which is ready for use to be administered simultaneously, concurrently, separately or sequentially. The components A and B may be administered independently of one another by the oral, intravenous, topical, local installations, intraperitoneal or nasal route. Preferably components A and B are both administered by the oral route or component A is administered by the oral route and component B is administered by the intravenous route.

Further, the present invention relates to a kit comprising

component A as described supra, particularly Compound A;
component B as described supra; particularly Compound B;
and, optionally,
component C: one or more, preferably one, further pharmaceutical agent(s),
in which optionally either or all of said components A, B and C in any of the above-mentioned combinations are in the form of a pharmaceutical composition which is ready for use to be administered simultaneously, concurrently, separately or sequentially. The components A and B, optionally C, may be administered independently of one another by the oral, intravenous, topical, local installations, intraperitoneal or nasal route.

In another embodiment the present invention relates to a kit, in which said components A and B, and optionally C, each are in the form of a pharmaceutical composition and, optionally, in which said component A is administered prior to component B and B is administered prior to component C.

In another embodiment the present invention relates to a kit, in which said components A and B, and optionally C, each are in the form of a pharmaceutical composition and, optionally, in which said component A is administered prior to component C and C is administered prior to component B.

In another embodiment the present invention relates to a kit, in which said components A and B, and optionally C, each are in the form of a pharmaceutical composition and in which said component B is administered prior to component A, and A is administered prior to component C.

In another embodiment the present invention relates to a kit, in which said components A and B, and optionally C, each are in the form of a pharmaceutical composition and in which said component B is administered prior to component C, and C is administered prior to component A.

### Method of Treating a Disease

The present invention also relates to:
the combination/pharmaceutical composition/kit of the invention as described herein for use in the treatment or prophylaxis of a disease, particularly for the treatment or prophylaxis of a hyper-proliferative disease;
the use of the combination/pharmaceutical composition/kit of the invention as described herein for the preparation of a medicament for the treatment or prophylaxis of a disease, particularly for the treatment or prophylaxis of a hyper-proliferative disease; and
a method of treatment or prophylaxis of a disease, particularly a hyper-proliferative disease, comprising administering to a mammal in need thereof, including a human, an amount of the combination/pharmaceutical composition/kit of the invention as described herein, which is effective for the treatment or prophylaxis of said disease, particularly of said hyper-proliferative disease.

Within the context of the present invention, the term “hyper-proliferative disease” includes, but is not limited to, e.g., psoriasis, keloids, and other hyperplasias affecting the skin, benign prostate hyperplasia (BPH), as well as malignant neoplasia. Examples of malignant neoplasia treatable with the compounds according to the present invention include solid and hematological tumors. Solid tumors can be exemplified by tumors of the breast, bladder, bone, brain, central and peripheral nervous system, colon, anum, endocrine glands (e.g. thyroid and adrenal cortex), esophagus, endometrium, germ cells, head and neck, kidney, liver, lung, larynx and hypopharynx, mesothelioma, ovary, pancreas, prostate, rectum, renal, small intestine, soft tissue, testis, stomach, skin, ureter, vagina and vulva. Malignant neoplasias include inherited cancers exemplified by Retinoblastoma and Wilms tumor. In addition, malignant neoplasias include primary tumors in said organs and corresponding secondary tumors in distant organs (“tumor metastases”). Hematological tumors can be exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, and cancers of unknown primary site as well as AIDS related malignancies.

Examples of breast cancer include, but are not limited to invasive ductal carcinoma, invasive lobular carcinoma, ductal carcinoma in situ, and lobular carcinoma in situ, particularly with bone metastases.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer.

The term “prostate cancer” as used herein means any histology type of prostate cancer including but not limited to acinar adenocarcinoma, ductal adenocarcinoma, transitional cell (or urothelial) cancer, squamous cell cancer, carcinoid, small cell cancer, sarcomas and sarcomatoid cancers, particularly acinar adenocarcinoma, metastatic hormone sensitive prostate cancer (mHSPC), castration resistant prostate cancer (CRPC). Preferably, the present invention relates to the treatment or prophylaxis of castration resistant prostate cancer (CRPC).

Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell.

Lymphomas, examples of which include, but are not limited to, AIDS-related lymphoma, chronic lymphocytic lymphoma (CLL), non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell lymphoma DLBCL, double-hit lymphoma and double-expressor lymphoma; anaplastic large cell lymphoma, B-cell lymphoma, cutaneous T-cell lymphoma, Burkitt's lymphoma, follicular lymphoma, hairy cell lymphoma, Hodgkin's disease, mantle cell lymphoma (MCL), lymphoma of the central nervous system, small lymphocytic lymphoma and chronic lymphocytic lymphoma and Sezary syndrome.

Sarcomas include, but are not limited to sarcoma of the soft tissue, osteosarcoma, malignant fibrous histiocytoma, lymphosarcoma, and rhabdomyosarcoma.

Leukemias, examples of which include, but are not limited to acute lymphoblastic leukemia, acute myeloid leukemia, (acute) T-cell leukemia, acute lymphoblastic leukemia, acute lymphocytic leukemia (ALL), acute monocytic leukemia (AML), acute promyelocytic leukemia (APL), bisphenotypic B myelomonocytic leukemia, chronic lymphocytic leukemia, chronic myelogenous leukemia, chronic myeloid leukemia (CML), chronic myelomonocytic leukemia (CMML), large granular lymphocytic leukemia, plasma cell leukemia, and also myelodysplastic syndrome (MDS), which can develop into an acute myeloid leukemia.

In particular, the present invention relates to a method for using the combinations of the present invention, in the treatment or prophylaxis of a hyper-proliferative disease, particularly lymphomas, such as AIDS-related lymphoma, cutaneous T-cell lymphoma, Burkitt lymphoma, Hodgkin's disease, lymphoma of the central nervous system, or non-Hodgkin's lymphoma (hereinafter abbreviated to “NHL”), particularly 1st line, 2nd line, relapsed, refractory, indolent or aggressive non-Hodgkin's lymphoma (NHL), in particular follicular lymphoma (hereinafter abbreviated to “FL”), chronic lymphocytic leukemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”). This method comprises administering to a mammal in need thereof, including a human, an amount of a combination of this invention, which is effective for the treatment or prophylaxis of a hyper-proliferative disease, in particular non-Hodgkin's lymphoma (hereinafter abbreviated to “NHL”), particularly 1st line, 2nd line, relapsed, refractory, indolent or aggressive non-Hodgkin's lymphoma (NHL), in particular follicular lymphoma (hereinafter abbreviated to “FL”), chronic lymphocytic leukemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”).

### Dose and Administration

Based upon standard laboratory techniques known to evaluate compounds useful for the treatment of hyper-proliferative diseases, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredients to be administered in the treatment of one of these conditions can vary widely according to such considerations as the particular component and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1,500 mg of active ingredient and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific combination employed, the age and general condition of the patient, time of administration, route of administration, rate of excretion of the drug, drug combinations, and the like. The desired mode of treatment and number of doses of a combination of the present invention or a pharmaceutically acceptable salt or ester or composition thereof can be ascertained by those skilled in the art using conventional treatment tests.

Further, suitable dose(s), administration regime(s) and administration route(s) for components A and B, and optionally C, may be readily determined by standard techniques known to the skilled person.

The dose(s), administration regime(s) and administration route(s) may have to be adapted according to, inter alia, the indication, the indication stage, the patient age and/or the patient gender, among other factors. Such adaptations can be readily determined by standard techniques known to the skilled person.

Components A and B, and optionally C, can be administered to a patient orally, topically, parenterally, rectally, by inhalation, and by injection. Administration by injection includes intravenous, intramuscular, subcutaneous, and parenterally as well as by infusion techniques. The agents can be administered by any of the conventional routes of administration for these compounds.

The preferred route of administration for component B, particularly Compound B, is typically orally and for component A, particularly Compound A, is typically intravenously, which is the same route of administration used for each agent alone.

Any of the compounds of component A described supra can be administered in combination with a compound of general formula (I) or (Ib) described supra, particularly with Compound A, by any of the mentioned routes of administration.

For administering components A and B by any of the routes of administration herein discussed, component A, particularly Compound A, can be administered simultaneously with component B, particularly Compound B. This can be performed by administering a single formulation which contains both the component A, particularly Compound A, and the component B, particularly Compound B. This can be also performed by administering the component A, particularly Compound A, and the component B, particularly Compound B, in independent pharmaceutical compositions at the same time to a patient.

In an embodiment of the invention component A, particularly Compound A, is administered prior to component B, particularly Compound B.

In a preferred embodiment of the invention component B, particularly Compound B, is administered prior to component A, particularly Compound A.

In another embodiment component B, particularly Compound B, is administered 1 day to 28 days, 7 to 28 days, 14 to 28 days, 21 to 28 days, 1 to 7 days, 7 to 14 days, 14 to 21 days, 18 to 24 days, 1 day to 21 days, 7 days to 21 days, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days prior to the administration of component A, particularly prior to the administration of Compound A.

In another embodiment component B, particularly Compound B, is administered 1 day to 28 days, 7 to 28 days, 14 to 28 days, 21 to 28 days, 1 to 7 days, 7 to 14 days, 14 to 21 days, 18 to 24 days, 1 day to 21 days, 7 days to 21 days, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days prior to the first administration of component A, particularly prior to the first administration of Compound A.

In another regimen of administration, component A, particularly Compound A, and component B, particularly Compound B, can be administered once or more times per day on the day of administration.

The following Examples describe the feasibility of the present invention, but not restricting the invention to these Examples only.

### DESCRIPTION OF FIGURES

FIG. 1:

Tumor growth of the human prostate cancer xenograft model 22Rv1 in male Fox Chase SCID mice, after treatment with Compound A in combination with Compound B upon different schedules in comparison to the respective monotherapies and control.

### EXPERIMENTAL SECTION

The following abbreviations are used in the Examples:

“Compound A” is an example of component A. The term “Compound A” (or “cpd. A” or “copanlisib”) means 2-amino-N-[7-methoxy-8-(3-morpholin-4-ylpropoxy)-2,3-dihydroimid-azo[1,2-c]quinazolin-5-yl]pyrimidine-5-carboxamide dihydrochloride. Compound A is published in international patent application PCT/EP2012/055600, published as WO 2012/136553, as the compound of Examples 1 and 2. It is a compound of structure:

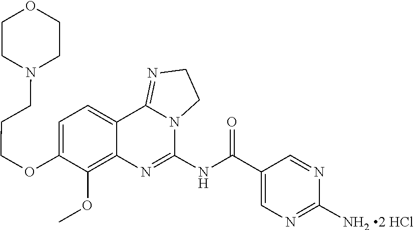

and is an example of component A as described and defined herein.

“Compound B” is an example of component B. The term “Compound B” means 2-[(3R)-3-methylmorpholin-4-yl]-4-(1-methyl-1H-pyrazol-5-yl)-8-(1H-pyrazol-5-yl)-1,7-naphthyridine. It is described in Example 111 of International Patent Application WO2016020320 (A1). Compound B is a compound of structure:

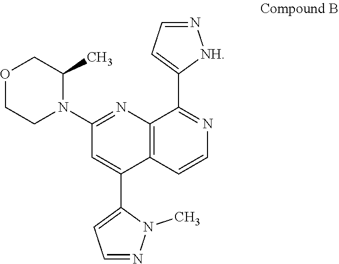 [SMILES: C[C@@H]1COCCN1c1cc(-c2ccnn2C)c2ccnc(-c3ccn[nH]3)c2n1]

### EXAMPLES

### Example 1

### Biological In Vitro Experiments

### 1.1 Test System

| | | | Plating |
|---|---|---|---|
| | | | cell |
| | | | number |
| Cell line | Tumor entity | Source | per well |
| NCI-H460 | non-small cell lung | ATCC HTB-144 | 600 |
| | carcinoma | | |
| HCT 116 | colon carcinoma | DSMZ ACC-581 | 700 |
| IGR-OV1 | endometrial carcinoma | NCI | 700 |
| SK-OV-3 | ovary carcinoma | NCI | 700 |
| Huh-7 | hepatocellular carcinoma | JCRB 0403 | 700 |
| PLC/PRF/5 | hepatocellular carcinoma | ATCC CRL-8024 | 700 |
| SNU-449 | hepatocellular carcinoma | ATCC CRL-2234 | 700 |
| MDA-MB-468 | mammary carcinoma | NCI | 700 |
| SUM-149 | mammary carcinoma | BiolVT | 700 |

Note:
ATCC, American Type Culture Collection, Manassas;
BiolVT, West Sussex;
DSMZ, German Collection of Microorganisms and Cell Cultures, Braunschweig;
JCRB, Japanese Cancer Research Resources Bank, Osaka;
NCI, National Cancer Institute, Bethesda

### 1.2 Study Design

| | final drug concentration (single compound | final vehicle |
|---|---|---|
| | treatments) | concentration |
| 1. Control | — | 0.3% dimethylsulfoxide |
| 2. PI3K | 1.0E−09M, 1.9E−09M, 3.7E−09M, 7.3E−09M, | 0.3% dimethylsulfoxide |
| inhibitor = | 1.4E−08M, 2.7E−08M, 5.2E−08M, 1.0E−07M | |
| Compound A | or | |
| | 1.0E−08M, 1.9E−08M, 3.7E−08M, 7.2E−08M, | |
| | 1.3E−07M, 2.7E−07M, 5.2E−07M, 1.0E−06M | |
| | or | |
| | 3.0E−08M, 5.8E−08M, 1.1E−07M, 2.1E−07M, | |
| | 4.1E−07M, 8.1E−07M, 1.6E−06M, 3.0E−06M | |
| 3. ATR kinase | 3.0E−09M, 5.8E−09M, 1.1E−08M, 2.1E−08M, | 0.3% dimethylsulfoxide |
| inhibitor = | 4.1E−08M, 8.1E−08M, 1.6E−07M, 3.0E−07M | |
| Compound B | | |

### 1.3 Methods and Parameters

Tumor cells were propagated in a humidified 37° C. incubator in their respective growth medium supplemented 10% fetal calf serum. For analysis of combination effects between Compound A and Compound B, cells were plated in 384-well plates at the cell numbers per well as indicated in 1.1 (Test system). After 24 h, cells were treated with a PI3K inhibitor (Compound A) and with a ATR inhibitor (Compound B) for single compound treatments (final concentrations see 1.2 (Study design)), and in nine different fixed-ratio combinations of Compound A (D1) and Compound B (D2) (0.9×D1+0.1×D2, 0.8×D1+0.2×D2, 0.7×D1+0.3×D2, 0.6×D1+0.4×D2, 0.5×D1+0.5×D2, 0.4×D1+0.6×D2, 0.3×D1+0.7×D2, 0.2×D1+0.8×D2, 0.1×D1+0.9×D2). Cell viability was assessed after 96 hour exposure with the Cell Titre-Glo Luminescent Cell Viability Assay (Promega). IC₅₀ values (inhibitory concentration at 50% of maximal effect) were determined by means of a 4 parameter fit on measurement data which were normalized to vehicle (DMSO) treated cells (=100%) and measurement readings taken immediately before compound exposure (=0%). IC₅₀ isobolograms were plotted with the actual concentrations of the two compounds on the x- and y-axis, and the combination index (CI) was calculated according to the median-effect model of Chou-Talalay [Chou T. C. Pharmacol. Rev. 58, 621, 2006]. A CI of <0.8 was defined as more than additive (synergistic) interaction, and a CI of >1.2 was defined as antagonistic interaction.

1.4 Results Calculated combination indices (CI₅₀) at IC₅₀ for ATR inhibitor Compound B plus PI3K inhibitor Compound A are summarized in Table 1, along with the mono-treatment IC₅₀ values and the concentrations required in combination to achieve the CI₅₀.

CI₅₀ interpretation code: CI₅₀<0.8, synergism; 0.8≤CI₅₀≤1.2, additivity; CI₅₀>1.2, antagonism.

TABLE 1
Calculated combination indices at IC₅₀ (CI₅₀) from proliferation assays of cell lines treated with
combinations of PI3K inhibitor Compound A and ATR inhibitor Compound B. Mono-treatment
IC₅₀ values and the concentrations required in combination of the two test compounds to achieve
the CI₅₀ are shown. All concentrations are given in mol/L. In case of synergism lowest CI₅₀
along with corresponding compound concentrations is presented, in case of antagonism, highest
CI₅₀ is given, and in case of additivity the CI₅₀ range is listed.

| | | IC_{50, mono} | IC_{50, mono} | Combination | | |
|---|---|---|---|---|---|---|
| | | Compound A | Compound B | Compound. A plus | | |
| Indication | Cell line | PI3K | ATR | Compound. B | | CI₅₀ |
| NSCLC | NCI-H460 | 6.3E−08M | 2.5E−07M | 7.2E−09 to | 2.2E−08 to | 0.90 to |
| | | | | 6.5E−08M | 1.9E−07M | 1.30 |
| Colon | HCT 116 | 9.5E−08M | 2.0E−07M | 7.4E−09 to | 2.6E−08 to | 0.96 to |
| | | | | 7.9E−08M | 2.0E−07M | 1.18 |
| Endometrium | IGR-OV1 | 1.9E−08M | 4.0E−08M | 1.4E−09 to | 5.3E−09 to | 0.97 to |
| | | | | 1.6E−08M | 3.8E−08M | 1.21 |
| Ovary | SK-OV-3 | 8.2E−08M | 1.3E−07M | 4.4E−09 to | 2.1E−08 to | 0.94 to |
| | | | | 6.3E−08M | 1.2E−07M | 1.09 |
| Liver | Huh-7 | 2.1E−08M | 5.1E−08M | 1.7E−09 to | 6.2E−09 to | 0.98 to |
| | | | | 1.9E−08M | 4.7E−08M | 1.11 |
| Liver | PLC/PRF/5 | 1.8E−07M | 6.7E−08M | 2.4E−07M | 1.6E−08M | 1.5 |
| Liver | SNU-449 | 1.0E−07M | 4.7E−07M | 2.6E−08M | 2.3E−07M | 0.75 |
| Breast | MDA-MB- | 2.8E−07M | 1.9E−07M | 5.8E−08 to | 8.9E−09 to | 1.01 to |
| | 468 | | | 2.7E−07M | 1.6E−07M | 1.21 |
| Breast | SUM-149 | 1.0E−08M | 1.8E−08M | 4.2E−09 to | 3.0E−10 to | 0.91 to |
| | | | | 9.0E−09M | 1.1E−08M | 1.17 |

### 1.5. Conclusions

In summary, in the present studies the combination of a PI3K inhibitor Compound A with an ATR inhibitor Compound B in proliferation assays of human carcinoma cells showed the utility of the present invention. The in vitro results showed additive and synergistic (CI₅₀<0.8) interaction of the PI3K inhibitor Compound A with the ATR inhibitor Compound B in the majority of the investigated cell lines.

### Example 2

### Biological In Vitro Experiments with Prostate Cancer Cell Lines

### 2.1 Test System

| | | | Plating |
|---|---|---|---|
| | | | cell |
| | | | number |
| Cell line | Tumor entity | Source | per well |
| VCaP | prostate carcinoma | ATCC CRL-2876 | 2400 |
| LNCaP | prostate carcinoma | DSMZ ACC-256 | 600 |
| C4-2B | prostate carcinoma | MD Anderson | 600 |
| | | Cancer Center | |
| 22Rv1 | prostate carcinoma | ATCC CRL-25O5 | 1200 |
| PC-3 | prostate carcinoma | DSMZ ACC-465 | 600 |

Note:
ATCC, American Type Culture Collection, Manassas;
DSMZ, German Collection of Microorganisms and Cell Cultures

### 2.2 Study Design

| | Drug concentration ranges used for | Final vehicle |
|---|---|---|
| | treatment | concentration |
| 1. Control | 0.1 nM R1881 (VCaP, 22Rv1, PC-3); | 0.2% |
| | 0.3 nM R1881 (LNCaP, C4-2B) | dimethylsulfoxide |
| 2. PI3K | 1.0E−09M, 3.16E−09M, 1.0E−08 M, | 0.2% |
| inhibitor = | 3.16E−08M, 1.0E−07M, 3.16E−07M, | dimethylsulfoxide |
| Compound A | 1.0E−06M | |
| 3. ATR kinase | 1.0E−09M, 3.16E−09M, 1.0E−08M, | 0.2% |
| inhibitor = | 3.16E−08M, 1.0E−07M, 3.16E−07M, | dimethylsulfoxide |
| Compound B | 1.0E−06M | |

### 2.3 Methods and Parameters

Tumor cells were propagated in a humidified 37° C. incubator in their respective growth medium supplemented with 10% fetal calf serum. For analysis of combination effects between Compound A and Compound B, tumor cells were plated in 384-well plates at the cell numbers per well indicated in 2.1 (Test system) in charcoal-stripped fetal calf serum. After 24 h, cells were treated with a fixed concentration of R1881 (see 2.2 (Study design)), and with a PI3K inhibitor (Compound A) and an ATR inhibitor (Compound B), either as single compound treatments or as seven different fixed-ratio combinations of Compound A/Compound B: 1/31.6, 1/10, 1/3.16, 1/1, 3.16/1, 10/1, 31.6/1. Cell viability was assessed after 144 hour exposure, except for PC-3 cells where measurement took place after 96 hour exposure, with the Cell Titre-Glo Luminescent Cell Viability Assay (Promega). IC₅₀ values (inhibitory concentration at 50% of maximal effect) were determined by means of a 4-parameter fit on measurement data which were normalized to data for treated cells at the end of the experiment (=100%) and for cells at day 0 immediately before compound exposure (C4-2B, 22Rv1 and PC-3 cells) or, in the case of VCaP and LNCaP, for cells at day 6 that were not treated with R1881 (=0%). IC₅₀ isobolograms were plotted with the actual concentrations of the two compounds on the x- and y-axis, and the combination index (CI) was calculated according to the median-effect model of Chou-Talalay [Chou T. C. Pharmacol. Rev. 58, 621, 2006]. Two separate experiments were performed to determine CI₅₀. A CI of <0.8 was defined as more than additive (synergistic) interaction, and a CI of >1.2 was defined as antagonistic interaction.

### 2.4 Results

Calculated combination indices (CI₅₀) at IC₅₀ for ATR inhibitor Compound B plus PI3K inhibitor Compound A are summarized in Table 2, along with the mono-treatment IC₅₀ values and the two combination ratios used to calculate the CI₅₀.

CI₅₀ interpretation code: CI₅₀<0.8, synergism; 0.8≤CI₅₀<1.2, additivity; CI₅₀>1.2, antagonism.

TABLE 2
Calculated combination indices CI₅₀ from proliferation assays of prostate
cancer cell lines treated with combinations of PI3K inhibitor (Compound A) and ATR
inhibitor (Compound B). Mono-treatment IC₅₀ values and the compound ratios of the
two test compounds used to calculate the CI₅₀ are shown.

| | | IC_{50, mono} | IC_{50, mono} | Combination | | |
|---|---|---|---|---|---|---|
| | | Compound A | Compound B | Mix ratio | | |
| Indication | Cell line | PI3K | ATR | Compound A | Compound B | CI₅₀ |
| Prostate | VCaP | 1.3E−8M | 1.6E−08M | 1 | 3.16 | 0.96 to 1.07 |
| carcinoma | | | | 1 | 1 | |
| Prostate | LNCaP | 8.0E−9M | 1.5E−08M | 1 | 1 | 0.94 to 1.04 |
| carcinoma | | | | 1 | 3.16 | |
| Prostate | C4-2B | 9.2E−08M | 3.5E−08M | 3.16 | 1 | 1.21 to 1.27 |
| carcinoma | | | | 10 | 1 | |
| Prostate | 22Rv1 | 5.0E−08M | 3.7E−08M | 3.16 | 1 | 0.63 to 0.66 |
| carcinoma | | | | 1 | 1 | |
| Prostate | PC-3 | 1.0E−07M | 1.3E−07M | 1 | 1 | 1.05 to 1.06 |
| carcinoma | | | | 1 | 3.16 | |

### 2.5. Conclusions

In summary, in the present studies the combination of a PI3K inhibitor Compound A with an ATR inhibitor Compound B in proliferation assays of human prostate carcinoma cells showed the utility of the present invention. The in vitro results showed additive and synergistic (CI₅₀<0.8) interaction of the PI3K inhibitor Compound A with the ATR inhibitor Compound B 4 out of 5 investigated prostate cancer cell lines.

### Example 3

### Biological In Vitro Experiments with Ovary Cancer Cell Lines

### 3.1 Test System

| | | | Plating |
|---|---|---|---|
| | | | cell |
| | | | number |
| Cell line | Tumor entity | Source | per well |
| A2780 | ovary carcinoma | ECACC 93112519 | 900 |
| OVCAR-3 | ovary carcinoma | NCI 0507262 | 1500 |

Note:
ECACC, European Collection of Cell Cultures, UK;
NCI, National Cancer Institute, Bethesda

### 3.2 Study Design

| | final drug concentration (single | final vehicle |
|---|---|---|
| | compound treatments) | concentration |
| 1. Control | — | 0.3% |
| | | dimethylsulfoxide |
| 2. PI3K | 8.6E−12M, 2.6E−11M, | 0.3% |
| inhibitor = | 1.0E−10M, 3.16E−10M, | dimethylsulfoxide |
| Compound A | 1.0E−09M, 3.16E−09M, | |
| | 1.0E−08M, 3.16E−08M, | |
| | 1.0E−07M, 3.16E−07M, | |
| | 1.0E−06M, 3.16E−06M, | |
| | 1.0E−05M | |
| 3. ATR kinase | 8.6E−12M, 2.6E−11M, | 0.3% |
| inhibitor = | 1.0E−10M, 3.16E−10M, | dimethylsulfoxide |
| Compound B | 1.0E−09M, 3.16E−09M, | |
| | 1.0E−08M, 3.16E−08M, | |
| | 1.0E−07M, 3.16E−07M, | |
| | 1.0E−06M, 3.16E−06M, | |
| | 1.0E−05M | |

### 3.3 Methods and Parameters

The effects of combinations of the present invention were evaluated using combination index isobologram analysis for in vitro assessment. The efficacy parameters were the effects in a 72-hour growth assay. Tumor cells were propagated in a humidified 37° C. incubator in their respective growth medium (RPMI) supplemented with 10% fetal calf serum. For analysis of combination effects between Compound A and Compound B, cells were plated in 384-well plates in 30 μl complete medium at the cell numbers per well as indicated in 2.1 (Study design). After 24 h, baseline cell growth was measured in a control plate using the CellTiter-Glo® Luminescent Cell Viability Assay (Promega), while cells in parallel assay plates were treated with a PI3K inhibitor (Compound A) and with a ATR inhibitor (Compound B) for single compound treatments (final concentrations see 2.2 (Study design)), and in nine different fixed-ratio combinations of Compound A (D1) and Compound B (D2) (0.9×D1+0.1×D2, 0.8×D1+0.2×D2, 0.7×D1+0.3×D2, 0.6×D1+0.4×D2, 0.5×D1+0.5×D2, 0.4×D1+0.6×D2, 0.3×D1+0.7×D2, 0.2×D1+0.8×D2, 0.1×D1+0.9×D2). Cell viability was assessed after 72 hour exposure with the Cell Titre-Glo Luminescent Cell Viability Assay (Promega). IC₅₀ values (inhibitory concentration at 50% of maximal effect) were determined by means of a 4 parameter fit on measurement data which were normalized to vehicle (DMSO) treated cells (=100%) and measurement reading from the control plate taken immediately before compound exposure in the parallel assay plates (=0%). IC₅₀ isobolograms were plotted with the actual concentrations of the two compounds on the x- and y-axis, and the combination index (CI) was calculated according to the median-effect model of Chou-Talalay [Chou T. C. Pharmacol. Rev. 58, 621, 2006]. A CI of <0.8 was defined as more than additive (synergistic) interaction, and a CI of >1.2 was defined as antagonistic interaction.

### 3.4 Results

Calculated combination indices (CI₅₀) at IC₅₀ for ATR inhibitor plus PI3K inhibitor are summarized in Table 3, along with the mono-treatment IC₅₀ values and the concentrations required in combination to achieve the CI₅₀.

CI₅₀ interpretation code: CI₅₀<0.8, synergism; 0.8≤CI₅₀≤1.2, additivity; CI₅₀>1.2, antagonism.

Table 3: Calculated combination indices at IC₅₀ (CI₅₀) from proliferation assays of cell lines treated with combinations of PI3K inhibitor Compound A and ATR inhibitor Compound B. Mono-treatment IC₅₀ values and the concentrations required in combination of the two test compounds to achieve the CI₅₀ are shown. All concentrations are given in mol/L. In case of synergism lowest CI₅₀ along with corresponding compound concentrations is presented.

| | | IC_{50, mono} [M] | IC_{50, mono} [M] | Combination (proliferation) | | |
|---|---|---|---|---|---|---|
| Indication | Cell Line | Compound A | Compound B | A [M] plus B [M] | | CI₅₀ |
| Ovary | A2780 | 3.74E−08 | 6.82E−08 | 6.96E−09 | 2.78E−08 | 0.59 |
| carcinoma | | | | | | |
| Ovary | OVCAR-3 | 1.20E−07 | 1.65E−07 | 4.48E−08 | 6.72E−08 | 0.78 |
| carcinoma | | | | | | |

### 3.5. Conclusions

In summary, in the present studies the combination of a PI3K inhibitor Compound A with an ATR inhibitor Compound B in proliferation assays of human ovary carcinoma cells showed the utility of the present invention. The in vitro results showed synergistic (CI₅₀<0.8) interaction of the PI3K inhibitor Compound A with the ATR inhibitor Compound B in the investigated cell lines.

### Example 4

### In Vivo Transplantation of Tumor

### Test Systems

| Cell line | Tumor entity | Mutation | Source |
|---|---|---|---|
| 22Rv1 | Prostate | ATM^{K1101E}, BRCA2^{fs}, ERCC3^{del}, MSH2^{del}, | ATCC CRL-2505 |
| | Carcinoma | NBN^{R43Q}, PALB2^{V1123M}, TP53^{Q331R}, | |
| | | WRN^{K5Nfs}*¹⁵, PIK3CA^{Q546R}, ATR^{fs} | |
| SUM-149 | mammary | FANCD2^{K50N}, FANCI^{D515H}, BRCA1^{N723X}, | BiolVT |
| | carcinoma | TP53^{M237I} | |
| IGR-OV1 | endometrial | PIK3CA^{R38C}, ATM^{R248Q}, DDB1^{D1115N}, | NCI |
| | carcinoma | ERBB3^{K742E}, MSH3^{G539V; F780L}, PIK3CA*^{1069W}, | |
| | | PTEN^{Y155C}, TP53^{Y126CI} | |
| RI-l | B Cell | MYC deregulation, CREBBP, FOXO5, | DSMZ ACC 585 |
| | Lymphoma | CDK5, GLI2, GPR112, IL17RD, MAP3K1, | |
| | (ABC-DLBCL) | MAP3K14, MYH1, NEK3, NOTCH1, | |
| | | PIK3C2G, PLK1, PRKDC, RECQL4, TP53, | |
| | | UBE3B, VEGFC, YSK4, BCL2 (high CN) | |

ATCC = American Type Culture Collection, Manassas, USA;
BiolVT, West Sussex, United Kingdom;
NCI = National Cancer Institute, Bethesda, USA;
DSMZ = German Collection of Microorganisms and Cell Cultures, Germany;
ABC-DLBCL = Activated B Cell-like Diffuse Large B Cell Lymphoma

The anti-tumor activity of combination treatment of Compound A and Compound B was examined in murine xenotransplantation models of human cancer cell lines. For this purpose, mice were implanted subcutaneously with tumor cells: 22Rv1 prostate cancer cells in male Fox Chase SCID mice from Harlan laboratories (UK), SUM-149 breast cancer cells or IGR-OV1 endometrial cancer cells in female NMRI nude mice and Ri-1 B cell lymphoma cells in female NOD/SCID mice from Janvier Labs (France). When animals reached a mean tumor size of 30-35 mm² (22Rv1) or of 30-40 mm² (SUM-149, IGR-OV1, Ri-1), animals were randomized into treatment and control groups (n=10 animals/group) and treatment started with Compound A monotherapy (formulation: 5% Mannitol in Water; application route: i.v./intravenous; dose/schedule: 10 or 14 mg/kg once daily for 2 days on/5 days off), Compound B monotherapy (formulation: 60% PEG400, 10% Ethanol, 30% Water; application route: p.o./peroral; dose/schedule: 10 (dose 1) or 20 mg/kg (dose 2) twice daily for 3 days on/4 days off), and combination of Compound A and Compound B at the same doses/schedules as in the respective monotherapies. Three different combination schedules were tested: (Schedule 1) Compound A applied on days 1 and 2 each week, Compound B applied on days 1, 2 and 3 each week; (Schedule 2) Compound A applied on days 1 and 2 each week, Compound B applied on days 3, 4 and 5 each week; (Schedule 3) Compound A applied on days 4 and 5 each week, Compound B applied on days 1, 2 and 3 each week. The oral application volume was 10 ml/kg and the intravenous application volume 5 ml/kg. The time interval between two applications per day was 6-7 h. The treatment was ended as soon as the group had mean tumor area ≤225 mm². The tumor size and the body weight were determined three times weekly. Changes in the body weight compared to the initial body weight at the start of treatment were a measure of treatment-related toxicity (>10%=critical, stoppage in treatment until recovery, >20%=toxic, termination). The tumor area was detected by means of an electronic caliper gauge [length (mm)×width (mm)]. In vivo anti-tumor efficacy is presented as T/C ratio (Treatment/Control) calculated with tumor areas at day of termination of control group by the formula [(tumor area of treatment group at day x)−(tumor area of treatment group at day before first treatment)]/[(tumor area control group at day x)−(tumor area of control group at day before first treatment)]. Compounds having a T/C below 0.5 were defined as active (effective). Statistical analysis was assessed using SigmaStat software. A one-way analysis of variance was performed and differences to the control were compared by a pair-wise comparison procedure (Dunn's method). To evaluate the cooperativity of the combination of Compound A with Compound B expected additivity was calculated according to the Bliss model (C=A+B−A*B; wherein C is the expected T/C of the combination of drug A and drug B if they act additive, A is T/C of drug A, B is T/C of drug B). Excess >10% over the expected additive effect is assumed to indicate synergism of the two drugs, less than 10% of the expected additive effect is assumed to indicate antagonism (Bliss, C. I., The toxicity of poisons applied jointly. Ann. Appl. Biol. 26, 585-615, 1939)

### Results:

In the 22Rv1 prostate cancer model monotherapies of Compound A or Compound B showed weak to moderate and statistically significant improved anti-tumor efficacy compared to control at the day of control group termination.

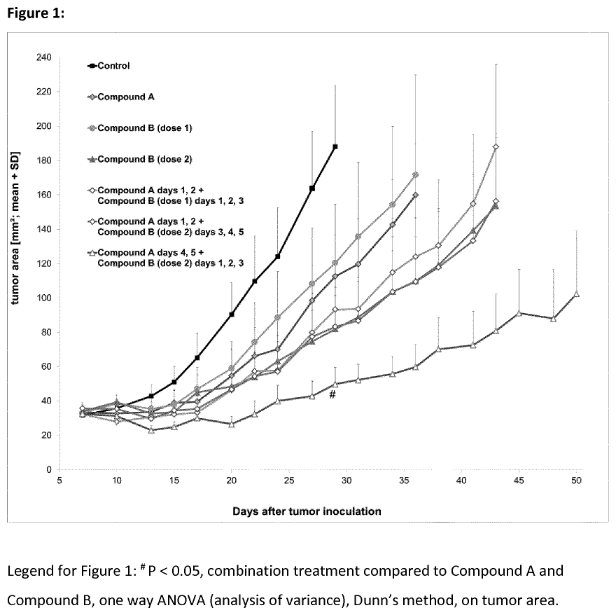

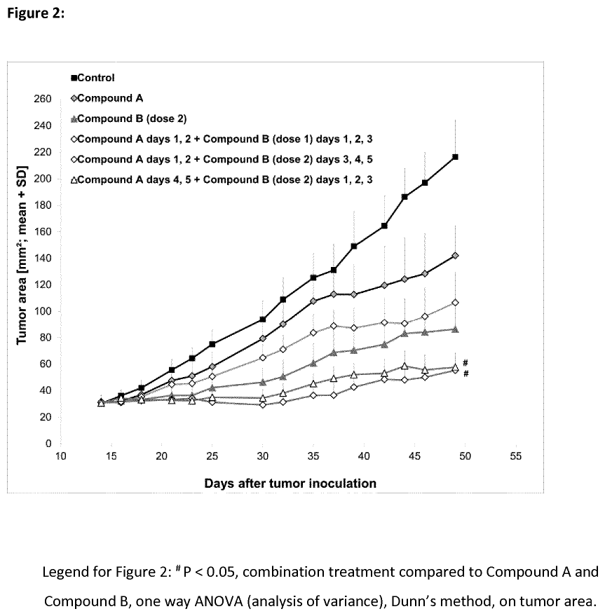

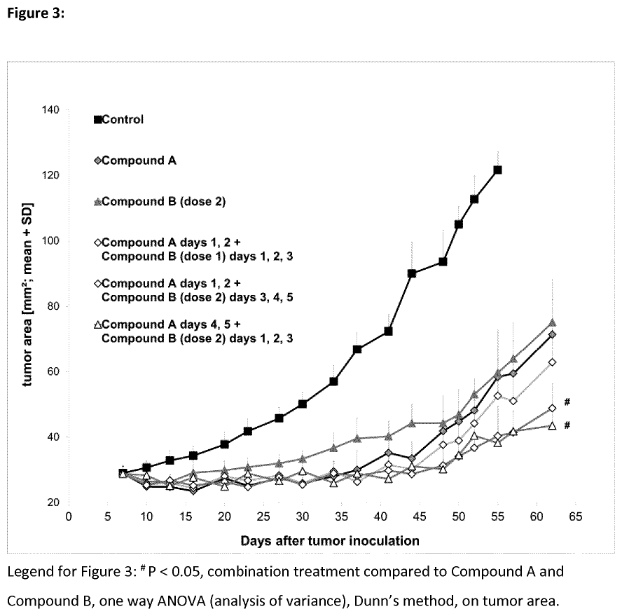

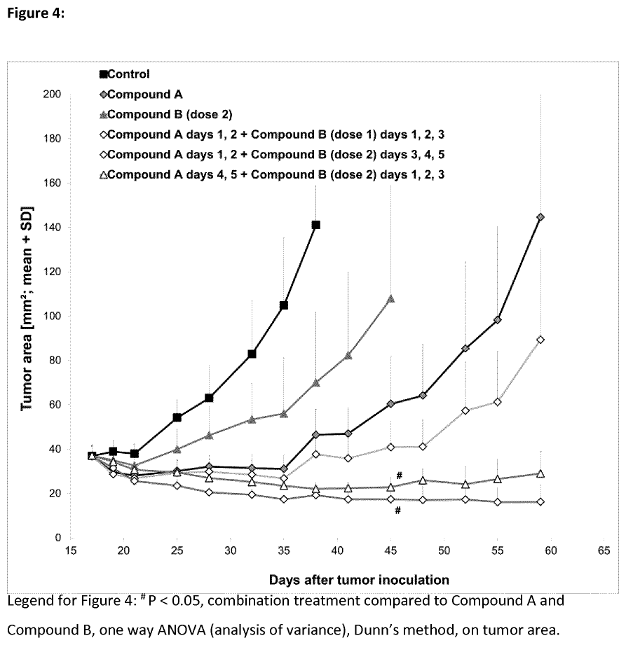

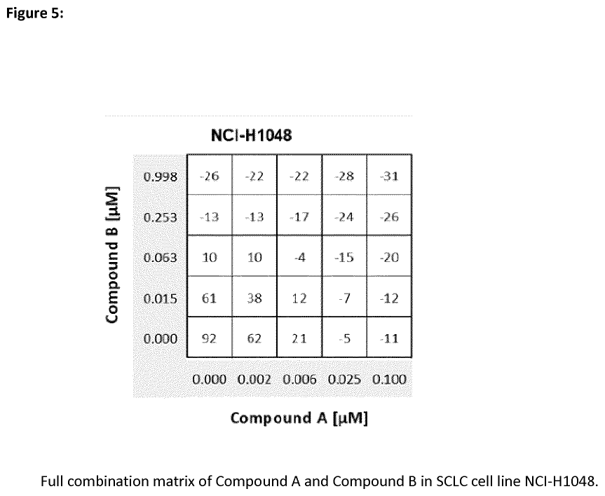

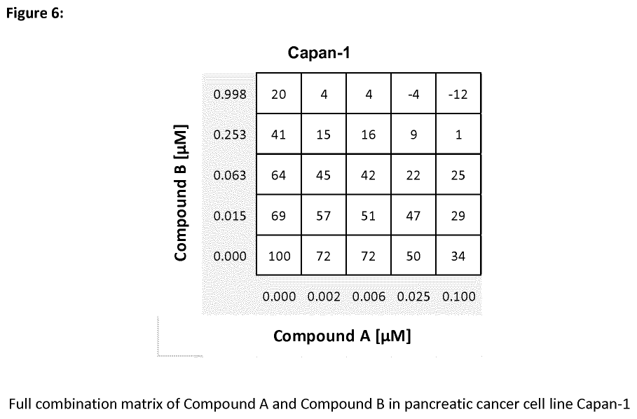

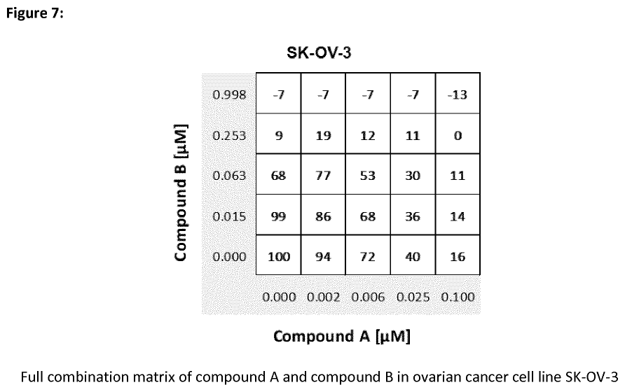

Combination of Compound A with Compound B in Schedule 3, when Compound B was applied before Compound A, enhanced anti-tumor efficacy of Compound B monotherapy at day 29, and demonstrated synergistic anti-tumor activity of Compound A and Compound B in this combination schedule, which was continued until study termination at day 50 after tumor inoculation and achieved strong and continuous tumor growth inhibition, as shown by statistically significant improvement of anti-tumor efficacy in comparison to Compound B alone, determined at day 29 after tumor inoculation (Table 4, FIG. 1). In contrast, combination of Compound A with Compound B in the other two tested schedules, when Compound A and Compound B were applied at the same time (Schedule 1) or when Compound A was applied before Compound B (Schedule 2), did not show significant effects on tumor growth inhibition, additivity or synergy. Overall tolerability of treatments was acceptable.

TABLE 4
Anti-tumor activity of Compound A and Compound B in monotherapy
and in combination in the human 22Rv1 prostate cancer
xenograft model in male Fox Chase SCID mice.

| | | | Excess over | |
|---|---|---|---|---|
| | | | Bliss additivity | |
| | | | [%] based on | Max. weight |
| Substance | Dosage | T/C^{a} | tumor size | loss^{b} (%) |
| Control | — | 1.00 | — | −2 |
| Compound A | 14 mg/kg, i.v., once daily, | 0.51* | — | −8 |
| | 2 days on/5 days off, | | | |
| | days 1, 2 each week | | | |
| Compound B | 10 mg/kg, p.o., twice daily, | 0.56* | — | −2 |
| (dose 1) | 3 days on/4 days off, | | | |
| | days 1, 2, 3 each week | | | |
| Compound B | 20 mg/kg, p.o., twice daily, | 0.31* | — | −6 |
| (dose 2) | 3 days on/4 days off, | | | |
| | days 3, 4, 5 each week | | | |
| Schedule (1) | 14 mg/kg, i.v., once daily, | 0.39* | — | −9 |
| Compound A + | 2 days on/5 days off, | | | |
| Compound B | days 1, 2 each week + | | | |
| (dose 1) | 10 mg/kg, p.o., twice daily, | | | |
| | 3 days on/4 days off, | | | |
| | days 1, 2, 3 each week | | | |
| Schedule (2) | 14 mg/kg, i.v., once daily, | 0.33* | — | −12 |
| Compound A + | 2 days on/5 days off, | | | |
| Compound B | days 1, 2 each week + | | | |
| (dose 2) | 20 mg/kg, p.o., twice daily, | | | |
| | 3 days on/4 days off, | | | |
| | days 3, 4, 5 each week | | | |
| Schedule (3) | 14 mg/kg, i.v., once daily, | 0.11*^{#} | 34 | −12 |
| Compound A + | 2 days on/5 days off, | | | |
| Compound B | days 4, 5 each week + | | | |
| (dose 2) | 20 mg/kg, p.o., twice daily, | | | |
| | 3 days on/4 days off, | | | |
| | days 1, 2, 3 each week | | | |

*P < 0.05 (compared to control)
^{#}P < 0.05 (compared to respective Compound A and Compound B monotherapies)
^{a}T/C = ratio of the relative tumor area of treatment versus control [(tumor area of treatment group at day x) − (tumor area of treatment group at day before first treatment)]/[(tumor area control group at day x) − (tumor area of control group at day before first treatment)].
^{b}Loss of body weight: Changes in body weight compared to the initial body weight at the start of treatment (>10% = critical, stoppage in treatment until recovery, >20% = toxic, termination).

The abbreviation QD means once per day, 2QD means twice per day, p.o. means peroral, i.v. means intravenous.

In the SUM-149 breast cancer model monotherapies of Compound A or Compound B showed weak to moderate and statistically significant improved anti-tumor efficacy compared to control at day 49 after tumor inoculation when all groups were terminated.

Combination of Compound A with Compound B in Schedule 2, when Compound A was applied before Compound B, enhanced anti-tumor efficacy of both Compound A and Compound B monotherapies, showing statistically significant improvement in comparison to Compound A or B alone at study end. Combination of Compound A with Compound B in Schedule 3, when Compound B was applied before Compound A, enhanced anti-tumor efficacy of Compound A and B monotherapy, showing statistically significant improvement in comparison to Compound A or B alone at study end. In contrast, combination of Compound A with Compound B in Schedule 1, when Compound A and Compound B were applied at the same time, did not show overall improvement of tumor growth inhibition compared to single agents. The overall tolerability of the treatments was acceptable (Table 5, FIG. 2).

TABLE 5
Anti-tumor activity of Compound A and Compound B in monotherapy
and in combination in the human SUM-149 breast cancer
xenograft model in female NMRI nude mice.

| | | | Excess over | |
|---|---|---|---|---|
| | | | Bliss additivity | |
| | | | [%] based on | Max. weight |
| Substance | Dosage | T/C^{a} | tumor size | loss^{b} (%) |
| Control | — | 1.00 | — | −5 |
| Compound A | 10 mg/kg, i.v., once daily, | 0.58* | — | −10 |
| | 2 days on/5 days off, | | | |
| | days 1, 2 each week | | | |
| Compound B | 20 mg/kg, p.o., twice daily, | 0.32* | — | −4 |
| (dose 2) | 3 days on/4 days off, | | | |
| | days 1, 2, 3 each week | | | |
| Schedule (1) | 10 mg/kg, i.v., once daily, | 0.39* | — | −10 |
| Compound A + | 2 days on/5 days off, | | | |
| Compound B | days 1, 2 each week + | | | |
| (dose 1) | 10 mg/kg, p.o., twice daily, | | | |
| | 3 days on/4 days off, | | | |
| | days 1, 2, 3 each week | | | |
| Schedule (2) | 10 mg/kg, i.v., once daily, | 0.11*^{#} | 10 | −10 |
| Compound A + | 2 days on/5 days off, | | | |
| Compound B | days 1, 2 each week + | | | |
| (dose 2) | 20 mg/kg, p.o., twice daily, | | | |
| | 3 days on/4 days off, | | | |
| | days 3, 4, 5 each week | | | |
| Schedule (3) | 10 mg/kg, i.v., once daily, | 0.15*^{#} | 4 | −7 |
| Compound A + | 2 days on/5 days off, | | | |
| Compound B | days 4, 5 each week + | | | |
| (dose 2) | 20 mg/kg, p.o., twice daily, | | | |
| | 3 days on/4 days off, | | | |
| | days 1, 2, 3 each week | | | |

*P < 0.05 (compared to control)
^{#}P < 0.05 (compared to respective Compound A and Compound B monotherapies)
^{a}T/C = ratio of the relative tumor area of treatment versus control [(tumor area of treatment group at day x) − (tumor area of treatment group at day before first treatment)]/[(tumor area control group at day x) − (tumor area of control group at day before first treatment)].
^{b}Loss of body weight: Changes in body weight compared to the initial body weight at the start of treatment (>10% = critical, stoppage in treatment until recovery, >20% = toxic, termination).

The abbreviation QD means once per day, 2QD means twice per day, p.o. means peroral, i.v. means intravenous.

In the IGR-OV1 endometrial cancer model monotherapies of Compound A or Compound B showed good to moderate and statistically significant improved anti-tumor efficacy compared to control at the day of control group termination (day 55 after tumor inoculation). Combination of Compound A with Compound B in Schedule 2, when Compound A was applied before Compound B, enhanced anti-tumor efficacy of both Compound A and Compound B monotherapies, showing statistically significant improvement in comparison to Compound A alone at day 55 and to both Compound A and Compound B at study termination day 62 after tumor inoculation. Combination of Compound A with Compound B in Schedule 3, when Compound B was applied before Compound A, enhanced anti-tumor efficacy of Compound A and B monotherapy, showing statistically significant improvement in comparison to Compound A alone at day 55 and to both Compound A and Compound B at study termination day 62 after tumor inoculation. In contrast, combination of Compound A with Compound B in Schedule 1, when Compound A and Compound B were applied at the same time, did not show overall improvement of tumor growth inhibition compared to single agents. The overall tolerability of the treatments was acceptable (Table 6, FIG. 3).

TABLE 6
Anti-tumor activity of Compound A and Compound B in monotherapy
and in combination in the human IGR-OV1 endometrial cancer
xenograft model in female NMRI nude mice.

| | | | Excess over | |
|---|---|---|---|---|
| | | | Bliss additivity | |
| | | | [%] based on | Max. weight |
| Substance | Dosage | T/C^{a} | tumor size | loss^{b} (%) |
| Control | — | 1.00 | — | −3 |
| Compound A | 10 mg/kg, i.v., once daily, | 0.32* | — | −6 |
| | 2 days on/5 days off, | | | |
| | days 1, 2 each week | | | |
| Compound B | 20 mg/kg, p.o., twice daily, | 0.33* | — | −6 |
| (dose 2) | 3 days on/4 days off, | | | |
| | days 1, 2, 3 each week | | | |
| Schedule (1) | 10 mg/kg, i.v., once daily, | 0.26* | — | −8 |
| Compound A + | 2 days on/5 days off, | | | |
| Compound B | days 1, 2 each week + | | | |
| (dose 1) | 10 mg/kg, p.o., twice daily, | | | |
| | 3 days on/4 days off, | | | |
| | days 1, 2, 3 each week | | | |
| Schedule (2) | 10 mg/kg, i.v., once daily, | 0.12*^{#} | — | −13 |
| Compound A + | 2 days on/5 days off, | | | |
| Compound B | days 1, 2 each week + | | | |
| (dose 2) | 20 mg/kg, p.o., twice daily, | | | |
| | 3 days on/4 days off, | | | |
| | days 3, 4, 5 each week | | | |
| Schedule (3) | 10 mg/kg, i.v., once daily, | 0.10*^{#} | 2 | −12 |
| Compound A + | 2 days on/5 days off, | | | |
| Compound B | days 4, 5 each week + | | | |
| (dose 2) | 20 mg/kg, p.o., twice daily, | | | |
| | 3 days on/4 days off, | | | |
| | days 1, 2, 3 each week | | | |

*P < 0.05 (compared to control)
^{#}P < 0.05 (compared to respective Compound A and Compound B monotherapies at study end)
^{a}T/C = ratio of the relative tumor area of treatment versus control [(tumor area of treatment group at day x) − (tumor area of treatment group at day before first treatment)]/[(tumor area control group at day x) − (tumor area of control group at day before first treatment)].
^{b}Loss of body weight: Changes in body weight compared to the initial body weight at the start of treatment (>10% = critical, stoppage in treatment until recovery, >20% = toxic, termination).

The abbreviation QD means once per day, 2QD means twice per day, p.o. means peroral, i.v. means intravenous.

In the Ri-1 B cell lymphoma model monotherapies of Compound A or Compound B showed good and statistically significant improved anti-tumor efficacy compared to control at the day of control group termination (day 38 after tumor inoculation).

Combination of Compound A with Compound B in Schedule 2, when Compound A was applied before Compound B, enhanced anti-tumor efficacy of both Compound A and Compound B monotherapies, showing statistically significant improvement of anti-tumor efficacy in comparison to Compound A and Compound B at day 45 after tumor inoculation when Compound B monotherapy group was terminated. Combination of Compound A with Compound B in Schedule 3, when Compound B was applied before Compound A, enhanced anti-tumor efficacy of Compound A and B monotherapy, showing statistically significant improvement of anti-tumor efficacy in comparison to Compound A and Compound B at day 45 after tumor inoculation when Compound B monotherapy group was terminated. Both combination schedules 2 and 3 led to long lasting tumor growth stagnation until study termination at day 59 in contrast to all single agent treatments. Combination of Compound A with Compound B in Schedule 1, when Compound A and Compound B were applied at the same time, showed only slight improvement of tumor growth inhibition compared to Compound A alone. The overall tolerability of treatments was acceptable (Table 7, FIG. 4).

TABLE 7
Anti-tumor activity of Compound A and Compound B in
monotherapy and in combination in the human Ri-1 B
cell lymphoma xenograft model in female NOD/SCID mice.

| | | | Excess over | |
|---|---|---|---|---|
| | | | Bliss additivity | |
| | | | [%] based on | Max. weight |
| Substance | Dosage | T/C^{a} | tumor size | loss^{b} (%) |
| Control | — | 1.00 | — | −7 |
| Compound A | 10 mg/kg, i.v., once daily, | 0.09* | — | −11 |
| | 2 days on/5 days off, | | | |
| | days 1, 2 each week | | | |
| Compound B | 20 mg/kg, p.o., twice daily, | 0.32* | — | −8 |
| (dose 2) | 3 days on/4 days off, | | | |
| | days 1, 2, 3 each week | | | |
| Schedule (1) | 10 mg/kg, i.v., once daily, | 0.01* | — | −10 |
| Compound A + | 2 days on/5 days off, | | | |
| Compound B | days 1, 2 each week + | | | |
| (dose 1) | 10 mg/kg, p.o., twice daily, | | | |
| | 3 days on/4 days off, | | | |
| | days 1, 2, 3 each week | | | |
| Schedule (2) | 10 mg/kg, i.v., once daily, | −0.17*^{#} | 20 | −10 |
| Compound A + | 2 days on/5 days off, | | | |
| Compound B | days 1, 2 each week + | | | |
| (dose 2) | 20 mg/kg, p.o., twice daily, | | | |
| | 3 days on/4 days off, | | | |
| | days 3, 4, 5 each week | | | |
| Schedule (3) | 10 mg/kg, i.v., once daily, | −0.14*^{#} | 17 | −11 |
| Compound A + | 2 days on/5 days off, | | | |
| Compound B | days 4, 5 each week + | | | |
| (dose 2) | 20 mg/kg, p.o., twice daily, | | | |
| | 3 days on/4 days off, | | | |
| | days 1, 2, 3 each week | | | |

*P < 0.05 (compared to control)
^{#}P < 0.05 (compared to respective Compound A and Compound B monotherapies at day 45)
^{a}T/C = ratio of the relative tumor area of treatment versus control [(tumor area of treatment group at day x) − (tumor area of treatment group at day before first treatment)]/[(tumor area control group at day x) − (tumor area of control group at day before first treatment)].
^{b}Loss of body weight: Changes in body weight compared to the initial body weight at the start of treatment (>10% = critical, stoppage in treatment until recovery, >20% = toxic, termination).

The abbreviation QD means once per day, 2QD means twice per day, p.o. means peroral, i.v. means intravenous.

### Example 5

### In Vitro Evaluation of Combination Sensitivity

Combination synergy defined by CI is a measure of drug interaction. Another important property for a drug combination is to evaluate sensitivity, a measure of drug combination efficacy. Combination sensitivity captures distinct properties of a drug combination to avoid biased prioritization of drug combinations that are unable to kill cancer cells despite strong synergy. Sensitivity of a drug combination is defined as the level of treatment response measured in the unit of percentage cell viability or growth in a full matrix design.

5.1 Test system

| Cell Line | Indication | Source | Plating cell number | |
|---|---|---|---|---|
| SK-OV-3 | Ovary carcinoma | NCI-60 panel | 800 | cells/well |
| H1048 | SCLC = small cell | ATCC | 1000 | cells/well |
| | lung carcinoma | | | |
| CAPAN-1 | Pancreatic cancer | ATCC | 500 | cells/well |

NCI = see above;
ATCC = see Example 4 above

### 5.2 Study Design

The full matrix design in this study covers the dose ranges for Compound A from 0 to 0.1 μM (clinical achievable concentrations) and Compound B from 0 to 1 μM. Percentage of control (DMSO) in tumor cell growth assay in vitro is used for the assessment of combination effects.

### Methods and Parameters

The effects of combinations of the present invention were evaluated using full matrix of 2 drug combination assessment in in vitro tumor growth assay. The efficacy parameters were the effects in a 72-hour assay. Tumor cells were cultured in 37° C. incubator in their respective growth medium and serum according to the protocol from providers. For analysis of combination effects between Compound A and Compound B, cells were plated in 384-well plates in 30 μl complete medium at the cell numbers per well as indicated in 5.1 (Test system). After 24 h, baseline cell growth was measured in a control plate using the CellTiter-Glo® Luminescent Cell Viability Assay (Promega), while cells in parallel assay plates were treated with a PI3K inhibitor (Compound A) and with a ATR inhibitor (Compound B) for single compound treatments, and in sixteen different combinations of Compound A (final concentrations are 0.002-0.006-0.025-0.100 μM) and Compound B (final concentrations are 0.015-0.063-0.253-0.998 μM). Cell viability was assessed after 72 hour exposure with the Cell Titre-Glo Luminescent Cell Viability Assay (Promega). Percentage growth (mono- or combination treatment of Compound A and/or Compound B vs DMSO control (100%)) were determined after normalized with the value before compound exposure in the parallel assay plates (=0%).

5.3 Combination Benefits of Compound a and Compound B in Small Cell Lung Cancer Cell Line NCI-H1048

FIG. 5 shows combination matrix of Compound A and Compound B in a 72-hour proliferation assay with small cell lung cancer cell line NCI-H1048. Treatment of Compound A or Compound B led to tumor killing at high concentration (0.025 FM and 0.253 μM, respectively). By combination of Compound A and Compound B, tumor killing can be achieved at much lower concentrations of both, Compound A (25%) and Compound B (25%). This result suggests that combination therapy could lower the dose to achieve tumor regression in preclinical tumor models or in small cell lung cancer patients. In addition, the data shown in FIG. 5 also suggested that sequential combination may not only increase but also prolong the anti-tumor activity as adding Compound A to low dose Compound B (e.g. 0.015) could enhance the activity from moderate anti-proliferation effect (61% growth inhibition) to tumor killing at the clinical achievable concentration of Compound A. It is also the case when adding Compound B to low dose Compound A.

5.4 Combination Benefits of Compound a and Compound B in Pancreatic Cancer Cell Line Capan-1

FIG. 6 shows combination matrix of Compound A and Compound B in a 72-hour proliferation assay in pancreatic cancer cell line Capan-1. Treatment of Compound A or Compound B as single agent led to moderate anti-proliferation effects at high concentration (0.025 μM and 0.063 μM, respectively). Combination of Compound A and Compound B, tumor growth inhibition can be achieved at much lower concentrations of both, Compound A (25%) and Compound B (25%). The tumor killing effect can be achieved at high dose combination of Compound A and Compound B, indicating simultaneous combination might be more beneficial.

5.5 Combination Benefits of Compound a and Compound B in Ovarian Cancer

FIG. 7 shows combination matrix of Compound A and Compound B in a 72-hour proliferation assay with ovarian cancer cell lines SK-OV-3. Treatment of Compound A or Compound B as single agent showed anti-proliferative effects at high concentrations (0.025 FM and 0.253 μM, respectively). Combination of Compound A and Compound B at high concentrations slightly increased combination benefits, while adding Compound A to low dose Compound B could enhance the activity from moderate to strong anti-proliferation effects, indicating that sequential combination may increase and prolong the anti-tumor activity as both compounds are dosed intermittently in preclinical and clinical settings.

1: A combination of component A and component B, wherein component A comprises one or more compounds of formula (A1): 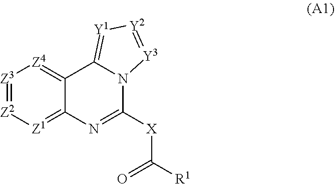
wherein
X is CR⁵R⁶ or NH;
Y¹ is CR³ or N;
Y² and Y³ are connected by a bond that is a single bond or a double bond,
with the proviso that when the bond is a double bond, Y² and Y³ are independently CR⁴ or N, and
when the bond is a single bond, Y² and Y³ are independently CR³R⁴ or NR⁴;
Z¹, Z², Z³ and Z⁴ are independently CH, CR² or N;
R¹ is aryl optionally having 1 to 3 substituents selected from R₁₁, C₃₋₈cycloalkyl optionally having 1 to 3 substituents selected from R¹¹,
C₁₋₆alkyl optionally substituted by
aryl, heteroaryl, C₁₋₆alkoxyaryl, aryloxy, heteroaryloxy or one or more halogen,
C₁₋₆alkoxy optionally substituted by
carboxy, aryl, heteroaryl, C₁₋₆alkoxyaryl, aryloxy, heteroaryloxy or one or more halogen,
or
a 3 to 15 membered mono- or bi-cyclic heterocyclic ring that is saturated or unsaturated, and contains 1 to 3 heteroatoms selected from the group consisting of N, O and S, and optionally having 1 to 3 substituents selected from R₁₁, wherein
R₁₁ is halogen, nitro, hydroxy, cyano, carboxy, amino, N—(C₁₋₆alkyl)amino, N-(hydroxyC₁₋₆alkyl)amino, N,N-di(C₁₋₆alkyl)amino, N—(C₁₋₆acyl)amino, N-(formyl)-N—(C₁₋₆alkyl)amino, N—(C₁₋₆alkanesulfonyl) amino, N-(carboxyC₁₋₆alkyl)-N—(C₁₋₆alkyl)amino, N—(C₁₋₆alkoxycabonyl)amino, N—[N,N-di(C₁₋₆alkyl)amino methylene]amino, N—[N,N-di(C₁₋₆alkyl)amino(C₁₋₆ alkyl)methylene]amino, N—[N,N-di(C₁₋₆alkyl)aminoC₂₋₆alkenyl]amino, aminocarbonyl, N—(C₁₋₆alkyl)aminocarbonyl, N,N-di(C₁₋₆alkyl)aminocarbonyl, C₃₋₈cycloalkyl, C₁₋₆ alkylthio, C₁₋₆alkanesulfonyl, sulfamoyl, C₁₋₆alkoxycarbonyl, N-arylamino wherein said aryl moiety is optionally having 1 to 3 substituents selected from R¹⁰¹, N-(aryl C₁₋₆alkyl)amino wherein said aryl moiety is optionally having 1 to 3 substituents selected from R¹⁰¹, arylC₁₋₆alkoxycarbonyl wherein said aryl moiety is optionally having 1 to 3 substituents selected from R¹⁰¹,
C₁₋₆alkyl optionally substituted by mono-, di- or tri-halogen, amino, N—(C₁₋₆alkyl)amino or N,N-di(C₁₋₆alkyl)amino, C₁₋₆alkoxy optionally substituted by mono-, di- or tri-halogen, N—(C₁₋₆alkyl)sulfonamide, or N-(aryl)sulfonamide,
or
a 5 to 7 membered saturated or unsaturated ring having 1 to 3 heteroatoms selected from the group consisting of O, S and N, and optionally having 1 to 3 substituents selected from R¹⁰¹, wherein
R¹⁰¹ is halogen, carboxy, amino, N—(C₁₋₆alkyl)amino, N,N-di(C₁₋₆alkyl)amino, aminocarbonyl, N—(C₁₋₆alkyl)aminocarbonyl, N,N-di(C₁₋₆alkyl)aminocarbonyl, pyridyl, C₁₋₆alkyl optionally substituted by cyano or mono- di- or tri-halogen,
or
C₁₋₆alkoxy optionally substituted by cyano, carboxy, amino, N—(C₁₋₆alkyl)amino, N,N-di(C₁₋₆alkyl)amino, aminocarbonyl, N—(C₁₋₆alkyl)aminocarbonyl, N,N-di(C₁₋₆alkyl)aminocarbonyl or mono-, di- or tri-halogen;
R² is hydroxy, halogen, nitro, cyano, amino, N—(C₁₋₆alkyl)amino, N,N-di(C₁₋₆alkyl)amino, N-(hydroxyC₁₋₆alkyl)amino, N-(hydroxyC₁₋₆alkyl)-N—(C₁₋₆alkyl)amino, C₁₋₆ acyloxy, aminoC₁₋₆ acyloxy, C₂₋₆alkenyl, aryl,
a 5-7 membered saturated or unsaturated heterocyclic ring having 1 to 3 heteroatoms selected from the group consisting O, S and N, and optionally substituted by hydroxy, C₁₋₆alkyl, C₁₋₆alkoxy, oxo, amino, aminoC1-6alkyl, N—(C₁₋₆alkyl)amino, N,N-di(C₁₋₆alkyl)amino, N—(C₁₋₆ acyl)amino, N—(C₁₋₆alkyl)carbonylamino, phenyl, phenylC1-6alkyl, carboxy, C₁₋₆alkoxycarbonyl, aminocarbonyl, N—(C₁₋₆alkyl)aminocarbonyl, or N,N-di(C₁₋₆alkyl)amino,
—C(O)— R²⁰,
C₁₋₆alkyl optionally substituted by R²¹, or C₁₋₆alkoxy optionally substituted by R²¹;
R²⁰ is C₁₋₆alkyl, C₁₋₆alkoxy, amino, N—(C₁₋₆alkyl)amino,
N,N-di(C₁₋₆alkyl)amino, N—(C₁₋₆acyl)amino, or a 5-7 membered saturated or unsaturated heterocyclic ring having 1 to 3 heteroatoms selected from the group consisting O, S and N, and optionally substituted by C₁₋₆ alkyl, C₁₋₆ alkoxy, oxo, amino, N—(C₁₋₆alkyl)amino, N,N-di(C₁₋₆alkyl)amino, N—(C₁₋₆ acyl)amino, phenyl, or benzyl,
R²¹ is cyano, mono-, di or tri-halogen, hydroxy, amino,
N—(C₁₋₆alkyl)amino, N,N-di(C₁₋₆alkyl)amino, N-(hydroxyC₁₋₆ alkyl)amino, N-(halophenylC₁₋₆alkyl)amino, aminoC₂₋₆alkylenyl, C₁₋₆alkoxy,
hydroxyC₁₋₆alkoxy, —C(O)—R²⁰¹, —NHC(O)—R²⁰¹, C₃₋₈cycloalkyl, isoindolino, phthalimidyl, 2-oxo-1,3-oxazolidinyl, aryl or a 5 or 6 membered saturated or unsaturated heterocyclic ring having 1 to 4 heteroatoms selected from the group consisting O, S and N optionally substituted by hydroxy, C₁₋₆alkyl, C₁₋₆alkoxy,
C₁₋₆alkoxycarbonyl, hydroxyC₁₋₆alkoxy, oxo, amino, aminoC₁₋₆alkyl, N—(C₁₋₆alkyl)amino, N,N-di(C₁₋₆alkyl)amino, N—(C₁₋₆ acyl)amino, or benzyl, wherein
R²⁰¹ is hydroxy, amino, N—(C₁₋₆alkyl)amino, N,N-di(C₁₋₆alkyl)amino, N-(halophenylC₁₋₆ alkyl)amino, C₁₋₆alkyl, aminoC₁₋₆alkyl, aminoC₂₋₆alkylenyl, C₁₋₆alkoxy, a 5 or 6 membered saturated or unsaturated heterocyclic ring having 1 to 4 heteroatoms selected from the group consisting O, S and N optionally substituted by hydroxy, C₁₋₆alkyl, C₁₋₆alkoxy, C₁₋₆alkoxycarbonyl,
hydroxyC₁₋₆alkoxy, oxo, amino, N—(C₁₋₆alkyl)amino, N,N-di(C₁₋₆alkyl)amino, N—(C₁₋₆acyl)amino or benzyl;
R³ is hydrogen, halogen, aminocarbonyl, or C₁₋₆alkyl optionally substituted by arylC₁₋₆alkoxy or mono-, di- or tri-halogen;
R⁴ is hydrogen or C₁₋₆alkyl;
R₅ is hydrogen or C₁₋₆alkyl; and
R⁶ is halogen, hydrogen or C₁₋₆alkyl;
or a stereoisomer, a hydrate, a solvate, or a pharmaceutically acceptable salt thereof; and
wherein component B comprises one or more ATR kinase inhibitor(s), or a stereoisomer, a tautomer, an N-oxide, a hydrate, a solvate, or a pharmaceutically acceptable salt thereof.

2: The combination according to claim 1, wherein component A is one or more compounds of formula (A2): 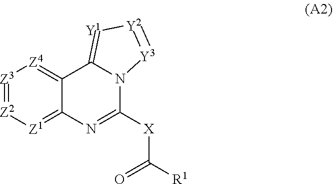
wherein:
X is CR⁵R⁶ or NH;
Y¹ is CR³ or N;
the bond between Y² and Y³ is a single bond or a double bond, with the proviso that when the bond between Y² and Y³ is a double bond, Y² and Y³ are independently CR⁴ or N, and
when the bond between Y² and Y³ is a single bond, Y² and Y³ are independently CR³R⁴ or NR⁴;
Z¹, Z², Z³ and Z⁴ are independently CH, CR² or N;
R¹ is aryl optionally having 1 to 3 substituents selected from R¹¹, C₃₋₈cycloalkyl optionally having 1 to 3 substituents selected from R¹¹,
C₁₋₆alkyl optionally substituted by aryl, heteroaryl, C₁₋₆alkoxyaryl, aryloxy, heteroaryloxy or one or more halogen,
C₁₋₆alkoxy optionally substituted by carboxy, aryl, heteroaryl, C₁₋₆alkoxyaryl, aryloxy, heteroaryloxy or one or more halogen,
or
a 3 to 15 membered mono- or bi-cyclic heterocyclic ring that is saturated or unsaturated, optionally having 1 to 3 substituents selected from R¹¹, and contains 1 to 3 heteroatoms selected from the group consisting of N, O and S, wherein
R₁₁ is halogen, nitro, hydroxy, cyano, carboxy, amino, N—(C₁₋₆alkyl)amino, N-(hydroxyC₁₋₆alkyl)amino, N,N-di(C₁₋₆alkyl)amino, N—(C₁₋₆acyl)amino, N-(formyl)-N—(C₁₋₆alkyl)amino, N—(C₁₋₆alkanesulfonyl) amino, N-(carboxyC₁₋₆alkyl)-N—(C₁₋₆alkyl)amino, N—(C₁₋₆alkoxycabonyl)amino, N—[N,N-di(C₁₋₆alkyl)amino methylene]amino, N—[N,N-di(C₁₋₆alkyl)amino (C₁₋₆alkyl)methylene]amino, N—[N,N-di(C₁₋₆alkyl)amino C₂₋₆alkenyl]amino, aminocarbonyl, N—(C₁₋₆alkyl)aminocarbonyl, N,N-di(C₁₋₆alkyl)aminocarbonyl, C₃₋₈cycloalkyl, C₁₋₆ alkylthio, C₁₋₆alkanesulfonyl, sulfamoyl, C₁₋₆alkoxycarbonyl,
N-arylamino wherein said aryl moiety is optionally having 1 to 3 substituents selected from R¹⁰¹, N-(aryl C₁₋₆alkyl)amino wherein said aryl moiety is optionally having 1 to 3 substituents selected from R¹⁰¹, aryl C₁₋₆alkoxycarbonyl wherein said aryl moiety is optionally having 1 to 3 substituents selected from R¹⁰¹,
C₁₋₆alkyl optionally substituted by mono-, di- or tri-halogen, amino, N—(C₁₋₆alkyl)amino or N,N-di(C₁₋₆alkyl)amino,
C₁₋₆alkoxy optionally substituted by mono-, di- or tri-halogen, N—(C₁₋₆alkyl)sulfonamide, or N-(aryl)sulfonamide,
or
a 5 to 7 membered saturated or unsaturated ring having 1 to 3 heteroatoms selected from the group consisting of O, S and N, and optionally having 1 to 3 substituents selected from R¹⁰¹, wherein
R¹⁰¹ is halogen, carboxy, amino, N—(C₁₋₆alkyl)amino, N,N-di(C₁₋₆alkyl)amino, aminocarbonyl, N—(C₁₋₆alkyl)aminocarbonyl, N,N-di(C₁₋₆alkyl)aminocarbonyl, pyridyl, C₁₋₆ alkyl optionally substituted by cyano or mono- di- or tri-halogen, or C₁₋₆alkoxy optionally substituted by cyano, carboxy, amino, N—(C₁₋₆alkyl)amino, N,N-di(C₁₋₆alkyl)amino, aminocarbonyl, N—(C₁₋₆alkyl)aminocarbonyl, N,N-di(C₁₋₆alkyl)aminocarbonyl or mono-, di- or tri-halogen;
R² is hydroxy, halogen, nitro, cyano, amino, N—(C₁₋₆alkyl)amino, N,N-di(C₁₋₆alkyl)amino, N-(hydroxyC₁₋₆alkyl)amino, N-(hydroxyC₁₋₆alkyl)-N—(C₁₋₆alkyl)amino, C₁₋₆ acyloxy, aminoC₁₋₆ acyloxy, C₂₋₆alkenyl, aryl,
a 5-7 membered saturated or unsaturated heterocyclic ring having 1 to 3 heteroatoms selected from the group consisting O, S and N, and optionally substituted by hydroxy, C₁₋₆alkyl, C₁₋₆alkoxy, oxo, amino, aminoC₁₋₆alkyl, N—(C₁₋₆alkyl)amino, N,N-di(C₁₋₆alkyl)amino, N—(C₁₋₆ acyl)amino, N—(C₁₋₆alkyl)carbonylamino, phenyl, phenylC1-6alkyl, carboxy, C₁₋₆alkoxycarbonyl, aminocarbonyl, N—(C₁₋₆alkyl)aminocarbonyl, or N,N-di(C₁₋₆alkyl)amino,
—C(O)—R²⁰, C₁₋₆ alkyl optionally substituted by R²¹, or C₁₋₆ alkoxy optionally substituted by R²¹, wherein
R²⁰ is C₁₋₆alkyl, C₁₋₆alkoxy, amino, N—(C₁₋₆alkyl)amino, N,N-di(C₁₋₆alkyl)amino, N—(C₁₋₆ acyl)amino, or a 5-7 membered saturated or unsaturated heterocyclic ring having 1 to 3 heteroatoms selected from the group consisting O, S and N, and optionally substituted by C₁₋₆alkyl, C₁₋₆ alkoxy, oxo, amino, N—(C₁₋₆alkyl)amino, N,N-di(C₁₋₆alkyl)amino, N—(C₁₋₆ acyl)amino, phenyl, or benzyl,
R²¹ is cyano, mono-, di or tri-halogen, hydroxy, amino, N—(C₁₋₆alkyl)amino, N,N-di(C₁₋₆alkyl)amino, N-(hydroxyC₁₋₆alkyl)amino, N-(halophenylC₁₋₆alkyl)amino, amino C₂₋₆alkylenyl, C₁₋₆alkoxy, hydroxyC₁₋₆alkoxy, —C(O)—R²⁰¹, —NHC(O)—R²⁰¹, C₃₋₈cycloalkyl, isoindolino, phthalimidyl, 2-oxo-1,3-oxazolidinyl, aryl or a 5 or 6 membered saturated or unsaturated heterocyclic ring having 1 to 4 heteroatoms selected from the group consisting O, S and N, and optionally substituted by hydroxy, C₁₋₆alkyl, C₁₋₆alkoxy, C₁₋₆alkoxycarbonyl, hydroxyC₁₋₆alkoxy, oxo, amino, aminoC₁₋₆alkyl, N—(C₁₋₆alkyl)amino, N,N-di(C₁₋₆alkyl)amino, N—(C₁₋₆ acyl)amino, or benzyl, wherein
R²⁰¹ is hydroxy, amino, N—(C₁₋₆alkyl)amino, N,N-di(C₁₋₆alkyl)amino, N-(halophenylC₁₋₆ alkyl) amino, C₁₋₆alkyl, aminoC₁₋₆ alkyl, aminoC₂₋₆ alkylenyl, C₁₋₆alkoxy, a 5 or 6 membered saturated or unsaturated heterocyclic ring having 1 to 4 heteroatoms selected from the group consisting O, S and N, and optionally substituted by hydroxy, C₁₋₆alkyl, C₁₋₆alkoxy, C₁₋₆alkoxycarbonyl, hydroxyC₁₋₆alkoxy, oxo, amino, N—(C₁₋₆alkyl)amino,N,N-di(C₁₋₆alkyl)amino, N—(C₁₋₆ acyl)amino or benzyl;
R³ is hydrogen, halogen, aminocarbonyl, or C₁₋₆alkyl optionally substituted by aryl C₁₋₆ alkoxy or mono-, di- or tri-halogen;
R⁴ is hydrogen or C₁₋₆alkyl;
R⁵ is hydrogen or C₁₋₆alkyl; and
R⁶ is halogen, hydrogen or C₁₋₆alkyl;
or a stereoisomer, a hydrate, a solvate, or a pharmaceutically acceptable salt thereof.

3: The combination according to claim 1, wherein component A is 2-amino-N-[7-methoxy-8-(3-morpholin-4-ylpropoxy)-2,3-dihydroimidazo[1,2-c]quinazolin-5-yl]pyrimidine-5-carboxamide or 2-amino-N-[7-methoxy-8-(3-morpholin-4-ylpropoxy)-2,3-dihydroimid-azo[1,2-c]quinazolin-5-yl]pyrimidine-5-carboxamide dihydrochloride;
or a stereoisomer, a hydrate, or a solvate thereof.

4: The combination according to claim 1, wherein component B comprises a compound selected from VX-803, VX-970, AZD-6738 and a compound of formula (I) 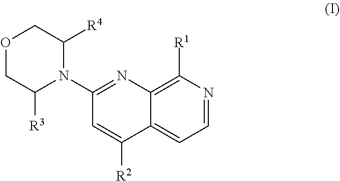
wherein:
R¹ is a group selected from: 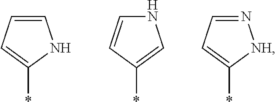
wherein * indicates the point of attachment of said group with the rest of the molecule;
R² is hydrogen, halogen, —NR⁷R₈, CN, C₁-C₆-alkyl, C₁-C₆-alkoxy, 3- to 10-membered heterocycloalkoxy, C₂-C₆-alkenyl, C₃-C₆-cycloalkyl, 3- to 10-membered heterocycloalkyl, 4- to 10-membered heterocycloalkenyl, phenyl, heteroaryl, —(CO)OR⁷, —(CO)NR⁷R⁸, —(SO₂)R⁹, —(SO)R⁹, —SR⁹, —(SO₂)NR⁷R⁹, —NR(SO₂)R⁹, —((SO)═NR¹¹)R¹⁰, —N═(SO)R⁹R¹⁰, —SiR₁₀R¹¹R¹², —(PO)(OR⁷)₂, —(PO)(OR⁷)R¹⁰ or —(PO)(R¹⁰)₂, wherein each C₁-C₆-alkyl, C₁-C₆-alkoxy, 3- to 10-membered heterocycloalkoxy, C₂-C₆-alkenyl, C₃-C₆-cycloalkyl, 3- to 10-membered heterocycloalkyl, phenyl or heteroaryl is optionally substituted, one or more times, independently from each other, with halogen, OH, —NR⁷R⁸, C₁-C₆-alkyl optionally substituted one or more times with hydroxyl or phenyl, C₁-C₆-haloalkyl, C₁-C₆-alkoxy, C₃-C₆-cycloalkyl, 3- to 6-membered heterocycloalkyl, phenyl, —(CO)OR⁷, —(CO)NR⁷R⁸, —NR⁷(CO)R¹⁰, —NR₈(CO)OR⁷, —NR⁸(CO) NR⁷R⁸, —(SO₂)R⁹, —(SO)R⁹, —SR⁹, —(SO₂)NR⁷R⁸, —NR⁷(SO₂)R⁹, —((SO)═NR¹¹)R¹⁰, —N═(SO)R⁹R¹⁰, —(PO)(OR⁷)₂, —(PO)(OR⁷)R¹⁰, —(PO)(R¹⁰)₂ or with a heteroaryl group which is optionally substituted, one or more times, with C₁-C₄-alkyl;
wherein each 4- to 10-membered heterocycloalkenyl is optionally substituted, one or more times, independently from each other, with C₁-C₄-alkyl;
R³ and R⁴ are independently hydrogen or methyl;
R⁷ and R⁸ are independently hydrogen, C₁-C₆-alkyl, C₃-C₆-cycloalkyl or phenyl, wherein phenyl is optionally substituted, one or more times, with halogen; or
R⁷ and R⁸ together with the nitrogen to which they are attached are a 4-, 5-, 6- or 7-membered cyclic amine group, which is optionally substituted, one or more times, independently from each other, with a substituent selected from C₁-C₆-alkyl, C₁-C₆-haloalkyl, said 4-, 5-, 6- or 7-membered cyclic amine group optionally containing one further heteroatom selected from the group consisting of O, N and S;
R⁹ is C₁-C₄-alkyl or phenyl, wherein each C₁-C₄-alkyl or phenyl is optionally substituted, one or more times, independently from each other, with R¹³;
R₁₀ is C₁-C₄-alkyl; or
R⁹ and R¹⁰ together with the sulphur atom to which they are attached, in case of —N═(SO)R⁹R¹⁰ group, represent a 5- to 8-membered heterocycloalkyl group;
R¹¹ is hydrogen, C₁-C₄-alkyl, —(CO)OR⁷, —(CO)NR⁷R⁸ or CN;
R¹² is hydrogen or C₁-C₄-alkyl;
R¹³ is halogen, OH, —NR⁷R⁸, CN, NO₂, C₁-C₆-alkyl, C₁-C₆-haloalkyl, C₁-C₆-alkoxy, C₁-C₆-haloalkoxy, C₂-C₆-alkenyl, C₃-C₆-cycloalkyl, —(CO)OR⁷ or —(CO)NR⁷R⁸;
or a stereoisomer, a tautomer, an N-oxide, a hydrate, a solvate, or a pharmaceutically acceptable salt thereof.

5: The combination according to claim 1, wherein component B is 2-[(3R)-3-methylmorpholin-4-yl]-4-(1-methyl-1H-pyrazol-5-yl)-8-(1H-pyrazol-5-yl)-1,7-naphthyridine or a stereoisomer, a tautomer, an N-oxide, a hydrate, a solvate, or a pharmaceutically acceptable salt thereof.

6: The combination according to claim 1, wherein:
component A is 2-amino-N-[7-methoxy-8-(3-morpholin-4-ylpropoxy)-2,3-dihydroimidazo[1,2-c]quinazolin-5-yl]pyrimidine-5-carboxamide or 2-amino-N-[7-methoxy-8-(3-morpholin-4-ylpropoxy)-2,3-dihydroimid-azo[1,2-c]quinazolin-5-yl]pyrimidine-5-carboxamide dihydrochloride, or a stereoisomer, a hydrate or a solvate thereof; and
component B is 2-[(3R)-3-methylmorpholin-4-yl]-4-(1-methyl-1H-pyrazol-5-yl)-8-(1H-pyrazol-5-yl)-1,7-naphthyridine, or a stereoisomer, a tautomer, an N-oxide, a hydrate, a solvate, or a pharmaceutically acceptable salt thereof.

7: The combination according to claim 1, wherein:
component A is 2-amino-N-[7-methoxy-8-(3-morpholin-4-ylpropoxy)-2,3-dihydroimidazo[1,2-c]quinazolin-5-yl]pyrimidine-5-carboxamide,
and
component B is 2-[(3R)-3-methylmorpholin-4-yl]-4-(1-methyl-1H-pyrazol-5-yl)-8-(1H-pyrazol-5-yl)-1,7-naphthyridine.

8: The combination according to claim 1, wherein:
component A is 2-amino-N-[7-methoxy-8-(3-morpholin-4-ylpropoxy)-2,3-dihydroimidazo[1,2-c]quinazolin-5-yl]pyrimidine-5-carboxamide dihydrochloride, and
component B is 2-[(3R)-3-methylmorpholin-4-yl]-4-(1-methyl-1H-pyrazol-5-yl)-8-(1H-pyrazol-5-yl)-1,7-naphthyridine.

9-10. (canceled)

11: A method of treatment or prophylaxis of a disease comprising administering to a mammal in need thereof an amount of the combination according to claim 1.

12: A pharmaceutical composition comprising the combination according to claim 1 and one or more pharmaceutically acceptable excipient(s).

13: A kit comprising the combination according to claim 1, in which both or either of components A and B are in the form of a pharmaceutical composition which may be administered simultaneously, concurrently, separately or sequentially.

14: The kit according to claim 13, in which component B is administered prior to component A.

15: The kit according to claim 13, in which component A is administered prior to component B.

16: The method of claim 11, wherein the disease is a hyper-proliferative disease.
